SEQUENCE LISTING (37 sequences)

SEQ ID NO: 1 - DNA, 339 bp, Mus sp.
  misc_feature (1)..(339): Polynucleotide sequence encoding 1181 light chain variable region
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaacgg

SEQ ID NO: 2 - DNA, 339 bp, Mus sp.
  misc_feature (1)..(339): Polynucleotide sequence encoding 1621 light chain variable region
gatgttgtgctgacccaaactccactctccctgcctgtcagtcttggagatcaagcctccatctcttgcagatctagtcagagccttgtacacagtaatggaagcacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataagacgg

SEQ ID NO: 3 - protein, 113 aa, Mus sp.
  MISC_FEATURE (1)..(113): 1181 light chain variable region amino acid sequence
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKR

SEQ ID NO: 4 - protein, 113 aa, Mus sp.
  MISC_FEATURE (1)..(113): 1621 light chain variable region amino acid sequence
DVVLTQTPLSLPVSLGDQASISCRSSQSLVHSNGSTYLHWYLQKPGQSPKLLIYKVSNRFSGVPDRFSGSGSGTDFTLKISRVEAEDLGVYFCSQSTHVPYTFGGGTKLEIKR

SEQ ID NO: 5 - protein, 16 aa, Mus sp.
  MISC_FEATURE (1)..(16): 1181 light chain CDR1 amino acid sequence
RSSQSLVHSNGNTYLH

SEQ ID NO: 6 - protein, 16 aa, Mus sp.
  MISC_FEATURE (1)..(16): 1621 light chain CDR1 amino acid sequence
RSSQSLVHSNGSTYLH

SEQ ID NO: 7 - protein, 16 aa, Mus sp.
  MISC_FEATURE (1)..(16): Light chain (generic for 1181 and 1621) CDR1 amino acid sequence
RSSQSLVHSNGXTYLH

SEQ ID NO: 8 - protein, 7 aa, Mus sp.
  MISC_FEATURE (1)..(7): 1181 and 1621 light chain CDR2 amino acid sequence
KVSNRFS

SEQ ID NO: 9 - protein, 9 aa, Mus sp.
  MISC_FEATURE (1)..(9): 1181 and 1621 light chain CDR3 amino acid sequence
SQSTHVPYT

SEQ ID NO: 10 - DNA, 357 bp, Mus sp.
  misc_feature (1)..(357): Polynucleotide sequence encoding 1181 heavy chain variable region
caggtccagctgcagcagtctggggcagagcttgtgaggtcaggggcctcagtcaagttgtcctgcacagcttctggcttcaacattaaagactactttatgcagtgggtgaagcagaggcctgaacagggcctggagtggattggatggattgatcctgagaatggtgaaactggatatgccccgaagttccagggcaaggccactatgactgcagacacagcctccaatacagcctacctgcaactcagcagcctgacatctgaggacactgccctgtattactgtaatgcaccttcggtcgtggctaaatacttcgatgtctggggcgcagggaccacggtcaccgtctcctca

SEQ ID NO: 11 - DNA, 357 bp, Mus sp.
  misc_feature (1)..(357): Polynucleotide sequence encoding 1621 heavy chain variable region
caggtccagctgcagcagtctggggcagagcttgtgaggtcaggggcctcagtcaagttgtcctgcacagcttctggcttcaacattaaagactactttgtgcagtgggtgaagcagaggcctgaacagggcctggagtggattggatggattgatcctgagaatggtgaaactggatatgccccgaagttccagggcaaggccactatgactgcagacacagcctccaatacagcctacctgcaactcagcagcctgacatctgaggacactgccctgtatttctgtaatgcaccttcggtagtggctaaatacttcgatgtctggggcgcagggaccacggtcaccgtctcctca

SEQ ID NO: 12 - protein, 119 aa, Mus sp.
  MISC_FEATURE (1)..(119): 1181 heavy chain amino acid sequence
QVQLQQSGAELVRSGASVKLSCTASGFNIKDYFMQWVKQRPEQGLEWIGWIDPENGETGYAPKFQGKATMTADTASNTAYLQLSSLTSEDTALYYCNAPSVVAKYFDVWGAGTTVTVSS

SEQ ID NO: 13 - protein, 118 aa, Mus sp.
  MISC_FEATURE (1)..(118): 1621 heavy chain amino acid sequence
QVQLQQSGAELVRSGASVKLSCTASGFNIKDYFVQWVKQRPEQGLEWGWIDPENGETGYAPKFQGKATMTADTASNTAYLQLSSLTSEDTALYFCNAPSVVAKYFDVWGAGTTVTVSS

SEQ ID NO: 14 - protein, 10 aa, Mus sp.
  MISC_FEATURE (1)..(10): 1181 heavy chain CDR1 amino acid sequence
GFNIKDYFMQ

SEQ ID NO: 15 - protein, 10 aa, Mus sp.
  MISC_FEATURE (1)..(10): 1621 heavy chain CDR1 amino acid sequence
GFNIKDYFVQ

SEQ ID NO: 16 - protein, 10 aa, Mus sp.
  MISC_FEATURE (1)..(10): Heavy chain CDR1 amino acid sequence (generic for 1181 and 1621)
GFNIKDYFXQ

SEQ ID NO: 17 - protein, 17 aa, Mus sp.
  MISC_FEATURE (1)..(17): 1181 and 1621 heavy chain CDR2 amino acid sequence
WIDPENGETGYAPKFQG

SEQ ID NO: 18 - protein, 10 aa, Mus sp.
  MISC_FEATURE (1)..(10): 1181 and 1621 heavy chain CDR3 amino acid sequence
PSVVAKYFDV

SEQ ID NO: 19 - protein, 28 aa, Homo sapiens
  MISC_FEATURE (1)..(28): Human ghrelin amino acid sequence
GSSFLSPEHQRVQQRKESKKPPAKLQPX

SEQ ID NO: 20 - protein, 16 aa, Mus sp.
  MISC_FEATURE (1)..(16): 4641 light chain CDR1 amino acid sequence
RSSQSLVHSDGNTYLD

SEQ ID NO: 21 - protein, 9 aa, Mus sp.
  MISC_FEATURE (1)..(9): 4641 light chain CDR3 amino acid sequence
SQSTYVSYT

SEQ ID NO: 22 - protein, 16 aa, Mus sp.
  MISC_FEATURE (1)..(16): 1451 light chain CDR1 amino acid sequence
RSSQSLVHSNGNTYLH

SEQ ID NO: 23 - protein, 9 aa, Mus sp.
  MISC_FEATURE (1)..(9): 1451 light chain CDR3 amino acid sequence
SQSTLVPYT

SEQ ID NO: 24 - protein, 10 aa, Mus sp.
  MISC_FEATURE (1)..(10): 4641 and 1451 heavy chain CDR1 amino acid sequence
AYTFTTYWMH

SEQ ID NO: 25 - protein, 17 aa, Mus sp.
  MISC_FEATURE (1)..(17): 4641 heavy chain CDR2 amino acid sequence
MIDPSNSDTWLNQKFKD

SEQ ID NO: 26 - protein, 17 aa, Mus sp.
  MISC_FEATURE (1)..(17): 1451 heavy chain CDR2 amino acid sequence
MIDPYNSETWLNQKFKD

SEQ ID NO: 27 - protein, 5 aa, Mus sp.
  MISC_FEATURE (1)..(5): 4641 and 1451 heavy chain CDR3 amino acid sequence
TGFDY

SEQ ID NO: 28 - protein, 16 aa, Mus sp.
  MISC_FEATURE (1)..(16): Light chain (generic for 4641, 1451, 1181, and 1621) CDR1 amino acid sequence
RSSQSLVHSXGXTYLX

SEQ ID NO: 29 - protein, 9 aa, Mus sp.
  MISC_FEATURE (1)..(9): Light chain (generic for 4641, 1451, 1181 and 1621) CDR3 amino acid sequence
SQSTXVXYT

SEQ ID NO: 30 - protein, 113 aa, Mus sp.
  MISC_FEATURE (1)..(113): 4641 light chain variable region amino acid sequence
DVVMTQTPLSLPVSLGDQASISCRSSQSLVHSDGNTYLHWYLQKPGQSPKLLIYKVSNRFSGVPDRFSGSGSGTDFTLKISRVEAEDLGVYFCSQSTYVSYTFGGGTKLEIKR

SEQ ID NO: 31 - protein, 113 aa, Mus sp.
  MISC_FEATURE (1)..(113): 1451 light chain variable region amino acid sequence
DVVLTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKR

SEQ ID NO: 32 - protein, 114 aa, Mus sp.
  MISC_FEATURE (1)..(114): 4641 heavy chain variable region amino acid sequence
QVQLQQSRPELVRPGASVKMSCRASAYTFTTYWMHWVRQRPGQGLEWIGMIDPSNSDTWLNQKFKDKATLNVDRSSNTAYMQLTSLTSEDSAVYYCARTGFDYWGQGTTLTVSS

SEQ ID NO: 33 - protein, 114 aa, Mus sp.
  MISC_FEATURE (1)..(114): 1451 heavy chain variable region amino acid sequence
QVHVKQSGPELVRPGASVKMSCKASAYTFTTYWMHWVKQRPGQGLEWIGMIDPYNSETWLNQKFKDKATLNVDRSSNTAYMKLSSLTSEDSAVYYCARTGFDYWGQGTTLTVSS

SEQ ID NO: 34 - DNA, 378 bp, Mus sp.
  misc_feature (1)..(378): 1451 LCVR polynucleotide
tctactccagcttgggcagatgttgtgctgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggtggaggcaccaagctggaaatcaaacgggctgatgcttcaccaactgta

SEQ ID NO: 35 - DNA, 360 bp, Mus sp.
  misc_feature (1)..(360): 1451 HCVR polynucleotide
caggtccacgtgaagcagtctgggcctgaactggtgaggcctggggcttcagtgaagatgtcctgcaaggcttcagcctataccttcaccacctactggatgcactgggtgaaacagaggcctggacaaggccttgagtggattggcatgattgatccttacaatagtgaaacttggttaaatcagaaattcaaggacaaggccacattgaatgtagacagatcctccaacacagcctacatgaagctcagcagcctgacatctgaggactctgcagtctattactgtgcaagaactggttttgactactggggccaaggcaccactctcacagtctcctcagccaaaacgacaccccca

SEQ ID NO: 36 - DNA, 378 bp, Mus sp.
  misc_feature (1)..(378): 4641 LCVR polynucleotide
tctactccagcttgggcagatgttgtgatgacccaaactccactctccctgcctgtcagtcttggagatcaagcctccatctcttgcagatctagtcagagccttgtacacagtgatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacatatgtttcgtacacgttcggaggggggaccaagctggaaataaaacgggctgatgctgcaccaactgta

SEQ ID NO: 37 - DNA, 360 bp, Mus sp.
  misc_feature (1)..(360): 4641 HCVR polynucleotide
caggtccagctgcagcagtctcggcctgaactggtgaggcctggggcttcagtgaagatgtcctgcagggcttcagcctataccttcaccacctactggatgcactgggtgagacagaggcctggacaaggccttgagtggattggcatgattgatccttccaatagtgatacttggttaaatcagaagttcaaggacaaggccacattgaatgtagacagatcctccaacacagcctacatgcagctcaccagcctgacatctgaggactctgcagtctattactgtgcaagaactggttttgactactggggccaaggcaccactctcacagtctcctcagccaaaacgacaccccca

(19) United States
(12) Patent Application Publication
(10) Pub. No.: US 2006/0233788 A1
(43) Pub. Date: Oct. 19, 2006

(54) Anti-ghrelin antibodies

(76) Inventors: Mark Louis Heiman, Indianapolis, IN (US); Kristine Kay Kikly, Fortville, IN (US); Joseph Vincent Manetta, Indianapolis, IN (US); Derrick Ryan Witcher, Fishers, IN (US)

Correspondence Address: ELI LILLY & COMPANY, PATENT DIVISION, P.O. BOX 6288, INDIANAPOLIS, IN, 46206-6288, US

(21) Appl. No.: 10/568,467

(22) PCT Filed: Sep. 2, 2004
(86) PCT No.: PCT/US04/25604

§ 371 Date: Feb. 14, 2006

Related U.S. Application Data
(60) Provisional application No. 60/500,496, filed on Sep. 5, 2003.
(60) Provisional application No. 60/572,249, filed on May 18, 2004.
(60) Provisional application No. 60/582,111, filed on Jun. 23, 2004.

(51) Int. Cl. A61K 39/395

(52) U.S. Cl. 424/133.1; 530/388.1

(57) ABSTRACT

Monoclonal antibodies, including humanized and chimeric antibodies, that bind acylated and unacylated human ghrelin are provided. Such monoclonal antibodies can be full-length, or an antigen-binding portion thereof, and are useful for neutralizing ghrelin activity, e.g., in a human subject suffering from a disorder in which ghrelin activity is detrimental.

### FIELD OF THE INVENTION

The present invention is in the field of medicine, particularly in the field of monoclonal antibodies against human ghrelin. More specifically the invention relates to monoclonal antibodies that bind both the acylated and unacylated forms of human ghrelin. The antibodies of the invention bind an antigenic epitope located within the peptide spanning amino acids 4-20 of human ghrelin. The antibodies of the invention may be murine, chimeric, or humanized antibodies, immunoconjugates of the antibodies or antigen-binding fragments thereof. The antibodies of the invention are useful for treatment of various diseases or disorders in humans wherein a decrease in ghrelin levels or activity contributes to a desirable therapeutic effect for disorders or conditions including obesity, obesity-related disorders such as NIDDM, eating disorders, gastric motility disorders, cardiovascular disease and cancer.

### BACKGROUND OF THE INVENTION

Ghrelin is a 28 amino acid peptide, a portion of which is acylated, typically with an n-octanoyl group, at the amino acid at position three (see SEQ ID NO: 19). The ghrelin hormone, when acylated, binds the growth hormone secretagogue receptor (GHS-R1a) in the pituitary resulting in release of growth hormone. The unacylated or “des-acyl” form of ghrelin does not bind the GHS-R1a receptor. (Kojima, et al., Nature 402:656-660, 1999). Ghrelin is also involved in energy balance, gastric motility and anxiety (Masuda, et al., Biochem Biophy Res Commun, 276:905-908, 2000; Asakawa, et al., Neuroendocrinology, 74:143-147, 2001).

The acylated form of ghrelin leads to fat deposition when administered to mice (Tschop, M. et al., Nature 407: 908-913, 2000). Ghrelin is synthesized primarily in the stomach and circulated in the blood. Ghrelin serum levels increase during food deprivation in animals (Kojima, et al., Nature 402:656-660, 1999), peak prior to eating (Cummings, et al., NEJM, 346:1623-1630, 2002) and decrease upon refeeding (Shiiya, et al., J. Clin. Endocrinol. Metab. 87:240-244, 2002). It has been shown that persons who underwent gastric bypass surgery and lost up to 36% of their body weight had greatly reduced ghrelin levels and loss of pre-meal peaks in ghrelin secretion. Persons with Prader-Willi syndrome, a genetic disorder that causes severe obesity with uncontrollable appetite, have extremely high levels of ghrelin. (Cummings, et al., NEJM, 346:1623-1630, 2002). These observations indicate that ghrelin plays a key role in motivating feeding. Additionally, ghrelin is believed to signal the hypothalamus when an increase in metabolic efficiency is required. (Muller, et al., Clin Endocrinol 55:461-467, 2001).

International patent publication number WO 01/07475 (EP 1197496) teaches the ghrelin amino acid sequence of various species, including human, and discloses that a fraction of ghrelin is acylated, typically with O-n-octanoic acid, at the third amino acid from the amino terminus, which is serine in native human ghrelin. WO 01/07475 also indicates that the amino terminal four amino acids of acylated ghrelin are essential for the GHSR1a receptor binding activity of ghrelin. The application further teaches antibodies directed against fatty acid-modified peptides of ghrelin, which peptides induce signal transduction, and the use of such antibodies for assaying or detecting ghrelin.

International patent publication number WO 01/87335 teaches the use of agents that specifically bind ghrelin, including anti-ghrelin antibodies, for the treatment of obesity.

Provisional patent application No. 60/475,708 filed Jun. 4, 2003; 60/491,352 filed Jul. 31, 2003 and 60/501,465 filed Sep. 9, 2004 all entitled “Anti-Ghrelin Antibodies” and assigned to Eli Lilly and Company, teach monoclonal anti-ghrelin antibodies which preferentially bind acylated human ghrelin with respect to unacylated human ghrelin and are useful for treatment of obesity and obesity-related disorders. Such antibodies include murine, chimeric and humanized antibodies.

International patent publication number WO 03/051389 teaches that administration of des-acyl ghrelin may prevent or reduce postprandial induction of insulin resistance by antagonizing some ghrelin actions and may reduce body weight in some patients.

Murakami, N. et al., administered to obese rats by intracerebroventricular injection a polyclonal anti-ghrelin antibody raised against the acylated amino-terminal eleven amino acids of rat ghrelin. The authors were able to demonstrate a subsequent decrease in both food intake and body weight by the rats. J. Endocrinology 174:283-288, 2002.

Obesity is a complex, chronic disease characterized by excessive accumulation of body fat and has a strong familial component. Obesity is generally the result of a combination of factors including genetic factors. Approximately 6% of the total population of the United States is morbidly obese. Morbid obesity is defined as having a body mass index of more than forty, or, as is more commonly understood, being more than one hundred pounds overweight for a person of average height. Obesity increases the risk of illness from about 30 serious medical conditions including osteoarthritis, Type II diabetes, hypertension, cancer and cardiovascular disease, and is associated with increases in deaths from all causes. Additionally, obesity is associated with depression and can further affect the quality of life through limited mobility and decreased physical endurance. Furthermore, it has been reported that some persons with eating disorders, such as anorexia nervosa, have altered ghrelin levels.

There are presently limited treatments for obesity. Current treatment options to manage weight include dietary therapy, increased physical activity and behavior therapy. Unfortunately, these treatments are largely unsuccessful with a failure rate reaching 95%. This failure may be due to the fact that the condition is strongly associated with genetically inherited factors that contribute to increased appetite, preference for highly caloric foods, reduced physical activity and increased lipogenic metabolism. This indicates that people inheriting these genetic traits are prone to becoming obese regardless of their efforts to combat the condition. Gastric bypass surgery is available to a limited number of obese persons. However, this type of surgery involves a major operation and cannot be modified readily as patient needs demand or change. Additionally, even this attempted remedy can sometimes fail (see, e.g., Kriwanek, Langenbecks Archiv. Fur Chirurgie, 38:70-74, 1995). Drug therapy options are few and of limited utility. Moreover, chronic use of these drugs can lead to tolerance, as well as side effects from their long term administration. And, when the drug is discontinued, weight often returns.

There is a tremendous therapeutic need for a means to treat obesity, obesity-related disorders, as well as other eating disorders. Due to its role in inducing feeding, ghrelin is a desirable target for therapeutic intervention. In particular, a monoclonal antibody against ghrelin may provide such a therapy. Of particular importance therapeutically is a humanized form of such a monoclonal antibody. Additionally, ghrelin is highly conserved in sequence and in function across species; therefore, not only may a monoclonal antibody of the invention be useful for the treatment of ghrelin-associated disorders in humans, but also in other mammals including, e.g. domestic animals (e.g., canine and feline), sports animals (e.g., equine) and food-source animals (e.g., bovine, porcine and ovine). An anti-ghrelin monoclonal antibody of the invention may be useful for the treatment of obesity and related disorders including, for example, Type II non-insulin dependent diabetes mellitus (NIDDM), Prader-Willi syndrome, eating disorders, hyperphagia, impaired satiety, cardiovascular disease and cancer.

### SUMMARY OF THE INVENTION

Monoclonal antibodies against human ghrelin (“hGhrelin”) that specifically bind both the acylated form of hGhrelin and the des-acyl (unacylated) form of hGhrelin are described in the present invention. Such antibodies are referred to herein as “anti-hGhrelin monoclonal antibodies” or “antibodies of the invention.” The monoclonal antibodies of the invention include murine monoclonal antibodies as well as chimeric monoclonal antibodies and humanized monoclonal antibodies. Preferably the antibodies of the invention exist in a homogeneous or substantially homogeneous population.

The invention provides anti-hGhrelin monoclonal antibodies which specifically bind an antigenic epitope localized within an antigenic peptide of both acylated hGhrelin and des-acyl hGhrelin, said antigenic peptide spanning amino acids 4-20 of human ghrelin (i.e., FLSPEHQRVQQRKESKK of SEQ ID NO: 19). Preferably said antibodies specifically bind acylated hGhrelin with no greater than six-fold or five-fold; more preferably no greater than four-fold or three-fold, and most preferably no greater than two fold or less difference than with which they specifically bind des-acyl hGhrelin as determined for example by ELISA assay or by K_{D} values in a Biacore assay (e.g. see Example 5). Antibodies of the invention specifically bind an antigenic peptide which peptide includes one or more of the amino acids that occur at positions 4, 5 and 6 of human ghrelin (i.e., “FLS” of SEQ ID NO: 19) and which spans 17, 16, 15, 14, 13, 12, 11, 10, 9, 8, 7 or 6 contiguous amino acids of human ghrelin, and is localized within a peptide spanning amino acids 4-20 of human ghrelin. The antigenic peptide may be conjugated to an immune potentiator, e.g., keyhole limpet hemocyanin (KLH). The antibodies of the invention disrupt or antagonize at least one in vitro or in vivo activity or biological property associated with acylated or des-acyl hGhrelin.

In one embodiment, an anti-hGhrelin monoclonal antibody of the invention comprises at least 1, 2 or 3, more preferably 4 or 5 peptides from peptides with a sequence selected from the group consisting of (a) SEQ ID NO: 5, 6, 7, 20, 22, or 28; (b) SEQ ID NO: 8, (c) SEQ ID NO: 9, 21, 23, or 29 (d) SEQ ID NO: 14, 15, 16, or 24; (e) SEQ ID NO: 17, 25, or 26 and (f) SEQ ID NO: 18 or 27. Preferably, the peptide with the sequence shown in SEQ ID NO: 5, 6, 7, 20, 22 or 28 when present in an antibody of the invention, is at light chain variable region (“LCVR”) CDR1. Preferably the peptide with the sequence shown in SEQ ID NO: 8, when present in an antibody of the invention, is at LCVR CDR2. Preferably the peptide with the sequence shown in SEQ ID NO: 9, 21, 23 or 29, when present in an antibody of the invention, is at LCVR CDR3. Preferably the peptide with the sequence shown in SEQ ID NO: 14, 15, 16, or 24, when present in an antibody of the invention, is at heavy chain variable region (“HCVR”) CDR1. Preferably the peptide with the sequence shown in SEQ ID NO: 17, 25 or 26, when present in an antibody of the invention, is at HCVR CDR2. Preferably the peptide with the sequence shown in SEQ ID NO: 18 or 27, when present in an antibody of the invention, is at HCVR CDR3. For approximate CDR locations within the LCVR or HCVR, see Tables 9-16 herein.

One embodiment provides an anti-hGhrelin monoclonal antibody comprising the 6 peptides with the sequences shown in SEQ ID NOs: 5, 8, 9, 14, 17 and 18. Preferably, the peptide with the sequence shown in SEQ ID NO: 5 is located at LCVR CDR1, the peptide with the sequence shown in SEQ ID NO: 8 is located at LCVR CDR2, the peptide with the sequence shown in SEQ ID NO: 9 is located at LCVR CDR3, the peptide with the sequence shown in SEQ ID NO: 14 is located at HCVR CDR1, the peptide with the sequence shown in SEQ ID NO: 17 is located at HCVR CDR2, and the peptide with the sequence shown in SEQ ID NO: 18 is located at HCVR CDR3.

Another embodiment provides an anti-hGhrelin monoclonal antibody comprising the 6 peptides with the sequences as shown in SEQ ID NOs: 6, 8, 9, 15, 17 and 18. Preferably, the peptide with SEQ ID NO: 6 is located at LCVR CDR1, the peptide with SEQ ID NO: 8 is located at LCVR CDR2, the peptide with SEQ ID NO: 9 is located at LCVR CDR3, the peptide with SEQ ID NO: 15 is located at HCVR CDR1, the peptide with SEQ ID NO: 17 is located at HCVR CDR2, and the peptide with SEQ ID NO: 18 is located at HCVR CDR3.

Another embodiment provides an anti-hGhrelin monoclonal antibody comprising the 6 peptides with the sequences as shown in SEQ ID NOs: 20, 8, 21, 24, 25 and 27. Preferably, the peptide with SEQ ID NO: 20 is located at LCVR CDR1, the peptide with SEQ ID NO: 8 is located at LCVR CDR2, the peptide with SEQ ID NO: 21 is located at LCVR CDR3, the peptide with SEQ ID NO: 24 is located at HCVR CDR1, the peptide with SEQ ID NO: 25 is located at HCVR CDR2, and the peptide with SEQ ID NO: 27 is located at HCVR CDR3.

Another embodiment provides an anti-hGhrelin monoclonal antibody comprising the 6 peptides with the sequences as shown in SEQ ID NOs: 22, 8, 23, 24, 26 and 27. Preferably, the peptide with SEQ ID NO: 22 is located at LCVR CDR1, the peptide with SEQ ID NO: 8 is located at LCVR CDR2, the peptide with SEQ ID NO: 23 is located at LCVR CDR3, the peptide with SEQ ID NO: 24 is located at HCVR CDR1, the peptide with SEQ ID NO: 26 is located at HCVR CDR2, and the peptide with SEQ ID NO: 27 is located at HCVR CDR3.

In another embodiment, an anti-hGhrelin monoclonal antibody of the invention comprises a light chain variable region (LCVR) comprising a peptide with the sequence shown in SEQ ID NO: 3, 4, 30 or 31. In another embodiment, an anti-hGhrelin monoclonal antibody of the invention comprises a heavy chain variable region (HCVR) comprising a peptide with the sequence shown in SEQ ID NO: 12, 13, 32 or 33. In another embodiment, an anti-hGhrelin monoclonal antibody of the invention comprises a LCVR comprising a peptide with the sequence shown in SEQ ID NO: 3, 4, 30 or 31 and further comprises a HCVR comprising a peptide with the sequence shown in SEQ ID NO: 12, 13, 32 or 33. An anti-hGhrelin monoclonal antibody of the invention may comprise a LCVR comprising a peptide with the sequence shown in SEQ ID NO: 3 and further comprise a HCVR comprising a peptide with the sequence shown in SEQ ID NO: 12. An anti-hGhrelin monoclonal antibody of the invention may comprise a LCVR comprising a peptide with the sequence shown in SEQ ID NO: 4 and further comprise a HCVR comprising a peptide with the sequence shown in SEQ ID NO: 13. An anti-hGhrelin monoclonal antibody of the invention may comprise a LCVR comprising a peptide with the sequence shown in SEQ ID NO: 30 and further comprise a HCVR comprising a peptide with the sequence shown in SEQ ID NO: 32. An anti-hGhrelin monoclonal antibody of the invention may comprise a LCVR comprising a peptide with the sequence shown in SEQ ID NO: 31 and further comprise a HCVR comprising a peptide with the sequence shown in SEQ ID NO: 33.

Preferably the LCVR CDR1 of an anti-hGhrelin monoclonal antibody of the invention comprises a peptide with the sequence shown in SEQ ID NO: 5, 6, 7, 20, 22 or 28. Preferably the LCVR CDR2 of an anti-hGhrelin monoclonal antibody of the invention comprises a peptide with the sequence shown in SEQ ID NO: 8. Preferably the LCVR CDR3 of an anti-hGhrelin monoclonal antibody of the invention comprises a peptide with the sequence shown in SEQ ID NO: 9, 21, 23 or 29. Preferably the HCVR CDR1 of an anti-hGhrelin monoclonal antibody of the invention comprises a peptide with the sequence shown in SEQ ID NO: 14, 15, 16 or 24. Preferably the HCVR CDR2 of an anti-hGhrelin monoclonal antibody of the invention comprises a peptide with the sequence shown in SEQ ID NO: 17, 25 or 26. Preferably the HCVR CDR3 of an anti-hGhrelin monoclonal antibody of the invention comprises a peptide with the sequence shown in SEQ ID NO: 18 or 27.

An anti-hGhrelin monoclonal antibody of the invention may further comprise a heavy chain constant region selected from the group consisting of IgG₁, IgG₂, IgG₃, IgG₄, IgA, IgE, IgM and IgD. Preferably the heavy chain constant region is IgG₄ or IgG₁. An anti-hGhrelin monoclonal antibody of the invention may further comprise a kappa or lambda chain constant region.

An anti-hGhrelin monoclonal antibody of the invention may comprise or consist of an intact antibody (i.e., full length), a substantially intact antibody, a Fab fragment, a F(ab′)₂ fragment or a single chain Fv fragment.

An anti-hGhrelin monoclonal antibody of the invention may comprise 1, 2, 3, 4, 5 or 6 peptides selected from peptides with a sequence selected from the group consisting of: (a) SEQ ID NO: 5, 6, 7, 20, 22 or 28 at LCVR CDR1 (b) SEQ ID NO: 8 at LCVR CDR2, (c) SEQ ID NO: 9, 21, 23 or 29 at LCVR CDR3, (d) SEQ ID NO: 14, 15, 16 or 24 at HCVR CDR1, (e) SEQ ID NO: 17,25 or 26 at HCVR CDR2; and (f) SEQ ID NO: 18 or 27 at HCVR CDR3 in which said peptide has 2 or 1 conservative amino acid substitutions and/or terminal deletions with respect to the sequence shown in said SEQ ID number.

In a preferred embodiment, an anti-hGhrelin monoclonal antibody of the invention is a chimeric antibody. In a more preferred embodiment, an anti-hGhrelin monoclonal antibody of the invention is a humanized antibody in which framework sequence and any constant region present in the antibody is of human origin or substantially of human origin. The humanized antibody is preferably a full-length antibody. Alternatively, the framework region, or a portion thereof, and any constant region present in the antibody my substantially originate from the genome of the animal in which the antibody is to be used as a therapeutic (e.g., canine, feline, equine, bovine, porcine and ovine). Furthermore, the antibody of the invention may be mutated to deimmunize the antibody.

In another embodiment, the invention provides an isolated nucleic acid molecule that encodes an LCVR of an antibody of the invention, and/or an HCVR of an antibody of the invention or an anti-hGhrelin monoclonal antibody of the invention. In one such embodiment, the LCVR polypeptide of an antibody of the invention is encoded by a polynucleotide comprising the sequence shown in SEQ ID NOs: 1, 2, 34 or 36. In another embodiment, the HCVR polypeptide of an antibody of the invention is encoded by a polynucleotide comprising the sequence shown in SEQ ID NOs: 10, 11, 35 or 37.

In another embodiment, the invention provides a vector, preferably a recombinant expression vector, comprising a polynucleotide encoding an anti-hGhrelin monoclonal antibody of the invention. Alternatively, a vector of the invention comprises a polynucleotide encoding an LCVR and/or an HCVR present in an anti-hGhrelin monoclonal antibody of the invention. The vector may comprise a polynucleotide with the sequence shown in SEQ ID NO: 1 and/or a polynucleotide with the sequence shown in SEQ ID NO: 10. In another embodiment, the vector may comprise a polynucleotide with the sequence shown in SEQ ID NO: 2 and/or a polynucleotide with the sequence shown in SEQ ID NO: 11. In another embodiment, the vector may comprise a polynucleotide with the sequence shown in SEQ ID NO: 34 and/or a polynucleotide with the sequence shown in SEQ ID NO: 35. In another embodiment, the vector may comprise a polynucleotide with the sequence shown in SEQ ID NO: 36 and/or a polynucleotide with the sequence shown in SEQ ID NO: 37. When both an LCVR and an HCVR DNA sequence are present in the same vector, they may be transcribed from one promoter to which they are both operably linked or they may be transcribed independently, each from a separate promoter to which it is operably linked. If the sequences encoding LCVR and HCVR are present in the same vector and transcribed from one promoter to which they are both operably linked, the LCVR may be 5′ to the HCVR or the LCVR may be 3′ to the HCVR, furthermore the LCVR and HCVR coding region in the vector may be separated by a linker sequence of any size or content, preferably such linker, when present, is a polynucleotide encoding an internal ribosome entry site.

In another embodiment, the invention provides a host cell comprising a nucleic acid molecule of the present invention. Preferably a “host cell of the invention” comprises one or more vectors or constructs comprising a nucleic acid molecule of the present invention. The host cell of the invention is a cell into which a vector of the invention has been introduced (e.g., via transformation, transduction, infection), said vector comprising a polynucleotide encoding a LCVR present in an antibody of the invention and/or a polynucleotide encoding a HCVR present in an antibody of the invention. The invention also provides a host cell into which two vectors of the invention have been introduced; one comprising a polynucleotide encoding a LCVR of an antibody of the invention and one comprising a polynucleotide encoding a HCVR present in an antibody of the invention and each operably linked to a promoter sequence. Preferably the vectors are integrated into the chromosomal DNA of the host cell. The host cell types include mammalian, bacterial, plant and yeast cells. Preferably the host cell is a CHO cell, a COS cell, a SP2/0 cell, a NSO cell, a yeast cell or a derivative of any preferred cell type.

In another embodiment, the invention provides a method of synthesizing an anti-hGhrelin monoclonal antibody of the invention comprising culturing a host cell of the invention (i.e., host cell that has been transformed, transduced or infected with a vector (or vectors) of the invention) in culture media such that an anti-hGhrelin monoclonal antibody of the invention or a fragment thereof is expressed in the cell. The antibody (or fragment thereof) is purified from the cell or preferably from the culture media in which said cell is grown.

The invention further embodies the process of producing an antibody of the invention by (i) immunizing a non-human animal, preferably a mouse or rat, with a peptide comprising 17, 16, 15, 14, 13, 12, 11, 10, 9, 8, 7 or 6 contiguous amino acids of the peptide spanning amino acid residues 4-20 of human ghrelin (see SEQ ID NO: 19) in which 1, 2 or 3 of said contiguous amino acids are selected from amino acids 4-6 of human ghrelin (“FLS” of SEQ ID NO: 19) wherein the immunogenic peptide is optionally conjugated to an immune potentiator, and (ii) isolating a monoclonal antibody from the immunized animal using any method known in the art, preferably by hybridoma synthesis. The anti-ghrelin antibodies are screened by any method available in the art (e.g., phage display, ribosome display, yeast display, bacterial display, ELISA assay) to identify an antibody that specifically binds both acylated hGhrelin and des-acyl hGhrelin. The invention further embodies a monoclonal antibody made by this process. Preferably said monoclonal antibody binds acylated hGhrelin with no greater than six-fold or five-fold; more preferably no greater than four-fold or three-fold, and most preferably no greater than two fold difference than with which it binds des-acyl hGhrelin as determined for example by ELISA assay or K_{D} values in a Biacore assay. It is contemplated that said antibody may be further altered into a chimeric antibody or a humanized antibody and still fall within the scope of the invention.

Various forms of the antibodies of the invention are contemplated herein. For example, an anti-hGhrelin monoclonal antibody of the invention may be a full length antibody (e.g., having a human immunoglobulin constant region) or an antibody fragment (e.g., a F(ab′)₂). It is understood that all such forms of the antibodies are encompassed herein within the term “antibody.” Furthermore, the antibody may be labeled with a detectable label, immobilized on a solid phase and/or conjugated with a heterologous compound (e.g., an enzyme or toxin) according to methods known in the art.

Diagnostic uses for antibodies of the invention are contemplated. In one diagnostic application, the invention provides a method for determining the presence of ghrelin protein comprising exposing a test sample suspected of containing the ghrelin protein to an anti-hGhrelin antibody of the invention and determining specific binding of the antibody to the sample. An anti-hGhrelin antibody of the invention may be used to determine the levels of ghrelin in test samples by comparing test sample values to a standard curve generated by binding said antibody to samples with known amounts of ghrelin. For diagnostic use, the invention provides a kit comprising an antibody of the invention and instructions for using the antibody to detect ghrelin protein in e.g. a test sample.

In another embodiment, the invention provides a pharmaceutical composition comprising an anti-hGhrelin monoclonal antibody of the invention. The pharmaceutical composition of the invention may further comprise a pharmaceutically acceptable carrier. In said pharmaceutical composition, the anti-hGhrelin monoclonal antibody of the invention is the active ingredient. Preferably the pharmaceutical composition comprises a homogeneous or substantially homogeneous population of an anti-hGhrelin monoclonal antibody of the invention. The composition for therapeutic use is sterile and may be lyophilized.

The invention provides a method of inhibiting ghrelin activity in a subject, preferably a human, in need thereof, whether that activity results from acylated ghrelin or des-acyl ghrelin or both, comprising administering a therapeutically effective amount, or prophylactically effective amount, of an anti-hGhrelin monoclonal antibody of the invention to said subject. The invention further provides a method of treating or preventing a disease or disorder ameliorated by the inhibition of signal transduction resulting from the binding of ghrelin to GHS-R1a which comprises administering to a subject or patient (e.g., a human) in need of such treatment or prevention a therapeutically or prophylactically effective amount of an antibody of the invention. As used herein, the term “treating or preventing a disease or disorder ameliorated by inhibition of signal transduction resulting from the binding of ghrelin to GHS-R1a” means relief from symptoms or conditions associated with abnormal ghrelin levels or benefited by a change in the existing ghrelin level, whether it be acylated ghrelin or des-acyl ghrelin. Diseases or disorders ameliorated by inhibition of signal transduction resulting from the binding of ghrelin to GHS-R1a may include, but are not limited to, obesity, NIDDM, Prader-Willi syndrome, eating disorders, hyperphagia, impaired satiety, anxiety, gastric motility disorders (including e.g., irritable bowel syndrome and functional dyspepsia), cancer, and cardiovascular disorders. The invention further provides a method for treating or preventing obesity and disorders related to obesity including for example, NIDDM, Prader-Willi syndrome, hyperphagia, impaired satiety, anxiety, gastric motility disorders (including e.g., irritable bowel syndrome and functional dyspepsia), cancer, and cardiovascular disorders in a human in need thereof by administering a therapeutically effective amount of an anti-hGhrelin monoclonal antibody of the invention.

The invention embodies an anti-hGhrelin monoclonal antibody of the invention for use in the manufacture of a medicament for administration to a human for the treatment of obesity and/or disorders related to obesity including for example, NIDDM, Prader-Willi syndrome, hyperphagia and impaired satiety in a human in need thereof by administering to said human a therapeutically effective or prophylactically effective amount of an anti-hGhrelin monoclonal antibody of the invention. The invention further embodies an anti-hGhrelin monoclonal antibody of the invention for use in the manufacture of a medicament for administration to a human for the treatment of anxiety, gastric motility disorders (including e.g., irritable bowel syndrome and functional dyspepsia), cancer, and cardiovascular disease in a human in need thereof by administering to said human a therapeutically effective amount of an anti-hGhrelin monoclonal antibody of the invention. The invention further embodies an anti-hGhrelin monoclonal antibody of the invention for use in the manufacture of a medicament for administration to other mammals including domestic animals, food source animals and sports animals for the treatment of the disorders listed above.

The invention embodies an article of manufacture comprising a packaging material and an antibody of the invention contained within said packaging material and wherein the packaging material comprises a package insert which indicates that the antibody neutralizes a ghrelin activity or decreases the level of active ghrelin.

TABLE 1
CDR Sequences of 1181 and 1621

| | | | |
|---|---|---|---|
| Domain | CDR1 | CDR2 | CDR3 |

| | | | |
|---|---|---|---|
| 1181 LCVR | RSSQSLVHSNGNTYLH | KVSNRFS | SQSTHVPYT |
| | (SEQ ID NO: 5) | (SEQ ID NO: 8) | (SEQ ID NO: 9) |
| 1621 LCVR | RSSQSLVHSNGSTYLH | KVSNRFS | SQSTHVPYT |
| | (SEQ ID NO: 6) | (SEQ ID NO: 8) | (SEQ ID NO: 9) |
| 4641 LCVR | RSSQSLVHSDGNTYLD | KVSNRFS | SQSTYVSYT |
| | (SEQ ID NO: 20) | (SEQ ID NO: 8) | (SEQ ID NO: 21) |
| 1451 LCVR | RSSQSLVHSNGNTYLH | KVSNRFS | SQSTLVPYT |
| | (SEQ ID NO: 22) | (SEQ ID NO: 8) | (SEQ ID NO: 23) |

| | | |
|---|---|---|
| CDR1 | CDR2 | CDR3 |

| | | | |
|---|---|---|---|
| 1181 HCVR | GFNIKDYFMQ | WIDPENGETGYAPKFQG | PSVVAKYFDV |
| | (SEQ ID NO: 14) | (SEQ ID NO: 17) | (SEQ ID NO: 18) |
| 1621 HCVR | GFNIKDYFVQ | WIDPENGETGYAPKFQG | PSVVAKYFDV |
| | (SEQ ID NO: 15) | (SEQ ID NO: 17) | (SEQ ID NO: 18) |
| 4641 HCVR | AYTFTTYWMH | MIDPSNSDTWLNQKFKD | TGFDY |
| | (SEQ ID NO: 24) | (SEQ ID NO: 25) | (SEQ ID NO: 27) |
| 1451 HCVR | AYTFTTYWMH | MIDPYNSETWLNQKFKD | TGFDY |
| | (SEQ ID NO: 24) | (SEQ ID NO: 26) | (SEQ ID NO: 27) |

### DETAILED DESCRIPTION OF THE INVENTION

Ghrelin was identified as the endogenous ligand of the growth hormone secretagogue receptor (GHS-R1a) (Kojima, M. et al. Nature 402:656-660, 1999). It is secreted foam multiple organs of the body but primarily from the stomach. The predominant active form of ghrelin present in humans is a 28 amino acid peptide acylated, typically with an n-octanoyl group, at the serine amino acid located at position 3. The unacylated, or “des-acyl” form of ghrelin does not bind GHS-R1a.

Recently ghrelin peptides with various modifications of the predominant form of ghrelin (SEQ ID NO: 19) have been identified in human stomach (Hosoda, H. et al., J. Biol. Chem. 278:64-70,2003). These minor forms include a 27 amino acid ghrelin peptide lacking the C-terminal Arg of the sequence that is shown in SEQ ID NO: 19 and ghrelin peptides decanoylated or decenoylated at position 3. The antibodies of the present invention bind both the 28 and 27 amino acid forms of hGhrelin (or even shorter forms when C-terminal deleted) both in the acylated and des-acyl form.

When it is necessary herein to refer specifically to the acylated form of ghrelin, it is referred to as “acylated ghrelin,” or “acylated hGhrelin” when referring specifically to human ghrelin. When referring specifically to the unacylated form of ghrelin, the term “des-acyl ghrelin” or “des-acyl hGhrelin” is used herein.

A full-length antibody as it exists naturally is an immunoglobulin molecule comprised of four peptide chains, two heavy (H) chains (about 50-70 kDa when full length) and two light (L) chains (about 25 kDa when full length) interconnected by disulfide bonds. The amino terminal portion of each chain includes a variable region of about 100-110 or more amino acids primarily responsible for antigen recognition. The carboxy-terminal portion of each chain defines a constant region primarily responsible for effector function.

Light chains are classified as kappa or lambda and characterized by a particular constant region. Heavy chains are classified as gamma, mu, alpha, delta, or epsilon, and define the antibody's isotype as IgG, IgM, IgA, IgD, and IgE, respectively. Each heavy chain type is characterized by a particular constant region.

Each heavy chain is comprised of a heavy chain variable region (herein “HCVR”) and a heavy chain constant region. The heavy chain constant region is comprised of three domains (CH1, CH2, and CH3) for IgG, IgD, and IgA; and 4 domains (CH1, CH2, CH3, and CH4) for IgM and IgE. Each light chain is comprised of a light chain variable region (herein “LCVR”) and a light chain constant region. The light chain constant region is comprised of one domain, CL. The HCVR and LCVR regions can be further subdivided into regions of hypervariability, termed complementarity determining regions (CDRs), interspersed with regions that are more conserved, termed framework regions (FR). Each HCVR and LCVR is composed of three CDRs and four FRs, arranged from amino-terminus to carboxy-terminus in the following order: FR1, CDR1, FR2, CDR2, FR3, CDR3, FR4. The assignment of amino acids to each domain is in accordance with well-known conventions [e.g., Kabat, “Sequences of Proteins of Immunological Interest,” National Institutes of Health, Bethesda, Md. (1987 and 1991) or Chothia numbering scheme as described in Al-Lazikani et al., J. Mol. Biol. 273:927-948, 1997, see also the internet site http:www.rubic.rdg.ac.uk/˜andrew/bioinf.org/abs. The functional ability of an antibody to bind a particular antigen is determined collectively by the six CDRs. However, even a single variable domain comprising only three CDRs specific for an antigen may have the ability to recognize and bind antigen, although at a lower affinity than a complete Fab.

The term “antibody,” in reference to an anti-hGhrelin antibody of the invention (or simply, “antibody of the invention”), as used herein, refers to a monoclonal antibody. A “monoclonal antibody” as used herein refers to a murine monoclonal antibody, a chimeric antibody or a humanized antibody. The term “monoclonal antibody” as used herein is not limited to antibodies produced through hybridoma technology. The term “monoclonal antibody” as used herein refers to an antibody that is derived from a single copy or clone, including any eukaryotic, prokaryotic, or phage clone, and not the method by which it is produced. A “monoclonal antibody” as used herein can be an intact (complete or full length) antibody, a substantially intact antibody, a portion or fragment of an antibody comprising an antigen-binding portion, e.g., a Fab fragment, Fab′ fragment or F(ab′)₂ fragment of a murine antibody or of a chimeric antibody or of a humanized antibody.

As used herein, the “antigen-binding portion” or “antigen-binding region” refers to that portion of an antibody molecule which contains the amino acid residues that interact with an antigen and confer on the antibody its specificity and affinity for the antigen. This antibody portion includes the “framework” amino acid residues necessary to maintain the proper conformation of the antigen-binding residues. Preferably, the CDRs of the antigen-binding region of the monoclonal antibodies of the invention will be of murine origin or substantially of murine origin. In other embodiments, the antigen-binding region can be derived from other non-human species such as rabbit, rat or hamster.

Furthermore, a “monoclonal antibody” as used herein can be a single chain Fv fragment that may be produced by joining the DNA encoding the LCVR and HCVR with a linker sequence. (See, Pluckthun, The Pharmacology of Monoclonal Antibodies, vol. 113, Rosenburg and Moore eds., Springer-Verlag, New York, pp 269-315, 1994). It is understood that regardless of whether fragments are specified, the term “antibody” as used herein includes such fragments as well as single chain forms. As long as the protein retains the ability to specifically bind its intended target (e.g., epitope or antigen), it is included within the term “antibody.” Antibodies may or may not be glycosylated and fall within the bounds of the invention.

A “monoclonal antibody” as used herein when referring to a population of antibodies, refers to a homogeneous or substantially homogeneous (or pure) antibody population (i.e., at least about 90%, 92%, 95%, 96%, more preferably at least about 97% or 98% or most preferably at least 99% of the antibodies in the population are identical and would compete in an ELISA assay for the same antigen. A monoclonal antibody of the invention may be expressed by a hybridoma, expressed recombinantly, or synthesized synthetically by means readily known in the art.

As used herein, “deimmunized” antibodies are antibodies with a reduced immunogenicity, i.e. antibodies that activate T-cells endogenous to the subject to a lesser extent than a comparable monoclonal, chimeric, humanized or human antibody. This may be necessary because monoclonal, chimeric or humanized antibodies can still elicit a T-cell response, due to presence of T-cell epitopes on the antibodies. In particular, complementarity-determining region residues or framework residues of antibodies can elicit an anti-idiotypic response by the host. This possibility of T-cell activation and response can also be exacerbated during the identification of higher affinity antibodies by affinity maturation. Examples of the ways in which decreased immunogenicity manifests itself include, but are not limited to, fewer T-cells being activated, or weaker binding of the T-cells to the immunogenic portion of the antibody. For preparation of deimmunized antibodies, epitopes recognized by T-cells are first identified. Briefly, the discrete number of MHC class II allotypes that occur in the population are identified and used to screen all possible 13-mer peptide fragments contained in the antibody to be deimmunized. Peptide binding grooves for each class II allotype are presented with peptides to determine, for each, a series of conformational binding scores that can be matched against a database of MHC Class II models. Biologically active T-cell epitopes achieve high binding scores while epitopes predicted to not engender a T-cell response give low binding scores. Modified sequences of the 13-mer peptides are subsequently designed to contain single amino acid substitutions to eliminate epitopes that can be recognized by the T-cells. Candidate variant antibodies are expressed and assayed, and lead deimmunized antibody candidates are selected.

The term “specific binding” or “specifically binds” as used herein refers to the situation in which one member of a specific binding pair will not show any significant binding to molecules other than its specific binding partner(s). The term is also applicable where e.g., an antigen-binding domain of an antibody of the invention is specific for a particular epitope that is carried by a number of antigens, in which case the specific antibody carrying the antigen-binding domain will be able to bind to the various antigens carrying the epitope.

The phrases “biological property” or “biological characteristic,” or the terms “biological activity” or “bioactivity,” in reference to an antibody of the present invention, are used interchangeably herein and include, but are not limited to, epitope/antigen affinity and specificity (e.g., anti-ghrelin monoclonal antibody binding to ghrelin), ability to antagonize an activity of the acylated or des-acyl ghrelin in vivo, in vitro, or in situ (e.g., growth hormone release), the in vivo stability of the antibody and the immunogenic properties of the antibody. Other identifiable biological properties or characteristics of an antibody recognized in the art include, for example, cross-reactivity, (ie., with non-human homologs of the targeted peptide, or with other proteins or tissues, generally), and ability to preserve high expression levels of protein in mammalian cells. The aforementioned properties or characteristics can be observed or measured using art-recognized techniques including, but not limited to ELISA, competitive ELISA, BIAcore® surface plasmon resonance analysis, in vitro and in vivo neutralization assays (see, e.g., Examples 2-5), and immunohistochemistry with tissue sections from different sources including human, primate, or any other source as the need may be.

The term “inhibit” or “inhibiting” means neutralizing, antagonizing, prohibiting, preventing, restraining, slowing, disrupting, stopping, or reversing progression or severity of that which is being inhibited, e.g., including, but not limited to a biological activity or property, a disease or condition.

The term “isolated” when used in relation to a nucleic acid or protein (e.g., an antibody), refers to a nucleic acid molecule or protein molecule that is identified and separated from at least one contaminant (nucleic acid or protein, respectively) with which it is ordinarily associated in its natural source. Isolated nucleic acid or protein is present in a form or setting that is different from that in which it is found in nature. In contrast, non-isolated nucleic acids or proteins are found in the state they exist in nature. Preferably, an “isolated antibody” is an antibody that is substantially free of other antibodies having different antigenic specificities (e.g., pharmaceutical compositions of the invention comprise an isolated antibody that specifically binds ghrelin substantially free of antibodies that specifically bind antigens other than ghrelin peptide).

The terms “Kabat numbering” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody (Kabat, et al., Ann. NY Acad. Sci. 190:382-93 (1971); Kabat, et al., Sequences of Proteins of Immunological Interest, Fifth Edition, U.S. Department of Health and Human Services, NIH Publication No. 91-3242 (1991)).

A polynucleotide is “operably linked” when it is placed into a functional relationship with another polynucleotide. For example, a promoter or enhancer is operably linked to a coding sequence if it affects the transcription of the sequence.

The term “neutralizing” or “antagonizing” in reference to an anti-hGhrelin (or anti-ghrelin) monoclonal antibody of the invention or the phrase “antibody that antagonizes (neutralizes) ghrelin activity” or “antagonizes (neutralizes) ghrelin” is intended to refer to an antibody whose binding to or contact with hGhrelin results in inhibition of a biological activity induced by acylated or des-acyl human ghrelin. Inhibition of hGhrelin biological activity can be assessed by measuring one or more in vitro or in vivo indicators of hGhrelin biological activity including, but not limited to, induction of weight loss, altered feeding, or inhibition of receptor binding (see WO 01/87335 for exemplary receptor binding assay) or signal transduction in a ghrelin-receptor binding assay. Indicators of ghrelin biological activity can be assessed by one or more of the several in vitro or in vivo assays known in the art. Preferably, the ability of an anti-ghrelin antibody to neutralize or antagonize ghrelin activity is assessed by use of the FLIPR assay as described in Example 4 herein.

The terms “individual,” “subject,” and “patient,” used interchangeably herein, refer to a mammal, including, but not limited to, murines, simians, humans, mammalian farm animals, mammalian sport animals, and mammalian pets; preferably the term refers to humans.

The term “K_{off},” as used herein, refers to the off rate constant for dissociation of an antibody from the antibody/antigen complex. The dissociation rate constant (K_{off}) of an anti-ghrelin monoclonal antibody can be determined by BIAcore® surface plasmon resonance as generally described in Example 5 herein. Generally, BIAcore® analysis measures real-time binding interactions between ligand (recombinant ghrelin peptide immobilized on a biosensor matrix) and analyte (antibodies in solution) by surface plasmon resonance (SPR) using the BIAcore system (Pharmacia Biosensor, Piscataway, N.J.). SPR can also be performed by immobilizing the analyte (antibodies on a biosensor matrix) and presenting the ligand in solution.

The term “K_{D},” as used herein, is refers to the equilibrium dissociation constant of a particular antibody-antigen interaction. For purposes of the present invention, K_{D} is determined as shown in Example 5. Antibodies that bind a particular epitope with high affinity have a K_{D} of 10⁻⁸ M or less, more preferably 10⁻⁹ M or less and most preferably 5×10⁻¹⁰ M or less.

The term “vector” includes a nucleic acid molecule capable of transporting another nucleic acid to which it has been linked including, but not limited to, plasmids vectors, yeast expression vectors, retroviral expression vectors and other viral vectors. Certain vectors are capable of autonomous replication in a host cell into which they are introduced while other vectors can be integrated into the genome of a host cell upon introduction into the host cell, and thereby, are replicated along with the host genome. Moreover, certain vectors are capable of directing the expression of genes to which a promoter within the vector is operably linked. Such vectors are referred to herein as “recombinant expression vectors” (or simply “expression vectors”) and exemplary vectors are well known in the art.

The term “host cell” includes an individual cell or cell culture that has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells tranfected, transformed, electroporated or infected in vivo or in vitro with a (one or more) recombinant vector or polynucleotide of the invention. A host cell comprises a recombinant vector of the invention either stably incorporated into the host chromosome or not and may also be referred to as a “recombinant host cell”. Preferred host cells for use in the invention are CHO cells (e.g., ATCC CRL-9096), NSO cells, SP2/0 cells and COS cells (ATCC e.g., CRL-1650, CRL-1651) and HeLa (ATCC CCL-2). Additional host cells for use in the invention include plant cells, yeast cells and other mammalian or bacterial cells.

The present invention relates to monoclonal antibodies that specifically bind both acylated hGhrelin and des-acyl hGhrelin. Antibodies of the invention neutralize a hGhrelin or a hGhrelin biological activity whether it be acylated hGhrelin or des-acyl hGhrelin or both. Activity inhibited is preferably (i) the binding of acylated hGhrelin to receptor GHS-R1a, (ii) signal transduction prompted by acylated hGhrelin binding GHS-R1a, (iii) binding of des-acyl hGhrelin to a binding partner with which it specifically binds, or (iv) signal transduction prompted by des-acyl hGhrelin binding a binding partner with which it specifically binds. Specific binding of anti-hGhrelin monoclonal antibodies of the invention (including antigen-binding portions thereof, and humanized monoclonal antibodies with like specificity) to hGhrelin, both acylated and des-acyl forms, allows said antibodies to be used as therapeutics or prophylactics for ghrelin-associated diseases and disorders, i.e., diseases or disorders which benefit from lowering or inhibiting a ghrelin bioactivity or the level of active ghrelin present in the subject.

Epitope Identification

The epitope to which the antibodies of the invention bind is localized within amino acids 4-20 of human ghrelin (SEQ ID NO: 19). The term “epitope” refers to that portion of any molecule capable of being recognized by and bound by an antibody at one or more of the antibody's antigen-binding regions. Epitopes often consist of a chemically active surface grouping of molecules such as amino acids or sugar side chains and have specific three dimensional structural characteristics as well as specific charge characteristics. By “inhibiting epitope” and/or “neutralizing epitope” is intended an epitope, which when specifically bound by an antibody, results in loss or decrease of a biological activity of the molecule or organism containing the epitope, in vivo, in vitro or in situ.

The term “epitope,” as used herein, further refers to a portion of a polypeptide having antigenic or immunogenic activity in an animal, preferably a mammal, e.g. a mouse or a human. The term “antigenic epitope,” as used herein, is defined as a portion of a polypeptide to which an antibody can specifically bind as determined by any method well known in the art, for example, by conventional immunoassays. Antigenic epitopes need not necessarily be immunogenic, but may be immunogenic. An “immunogenic epitope,” as used herein, is defined as a portion of a polypeptide that elicits an antibody response in an animal, as determined by any method known in the art. (See, e.g., Geysen et al., Proc. Natl. Acad. Sci. USA 81:3998-4002 (1983)).

The anti-hGhrelin monoclonal antibodies of the invention (“antibodies of the invention”) specifically bind to both the acylated and des-acyl forms of hGhrelin. The epitope to which they bind, i.e., the antigenic epitope, is localized to amino acids 4-20 of human ghrelin (see Examples 2-5 herein). The antigenic epitope comprises 17, 16, 15, 14, 13, 12, 11, 10, 9, 8, 7 or 6 contiguous amino acids of the peptide spanning amino acid residues 4-20 of human ghrelin (see SEQ ID NO: 19) in which 1, 2 or 3 of said contiguous amino acids are selected from amino acids 4-6 of human ghrelin. Preferably, the amino terminus of the antigenic epitope is amino acid 4, 5 or 6 of human ghrelin (“FLS” of SEQ ID NO: 19). Said antigenic epitope may possess additional human ghrelin residues outside of amino acids 420 of human ghrelin, but the monoclonal antibodies of the invention do not require these additional residues to specifically bind the antigenic epitope. Additional residues of hGhrelin outside of the amino acids 4-20 antigenic epitope may affect the conformational structure of the antigenic domain and thereby alter binding of an antibody of the invention to the antigenic epitope. The monoclonal antibodies of the invention bind acylated hGhrelin with no greater than six-fold or five-fold; more preferably no greater than four-fold or three-fold, and most preferably no greater than two fold difference than with which it binds des-acyl hGhrelin as determined e.g., by ELISA or K_{D} values in a Biacore® assay.

The domain spanning amino acids 4-20 (inclusive) of hGhrelin may also be used as an immunogenic epitope to generate monoclonal antibodies of the invention. This domain (i.e., amino acids 4-20 of SEQ ID NO: 19 “FLSPEHQRVQQRKESKK”) may be used to immunize a non-human animal, preferably a mouse. Then anti-hGhrelin antibodies are isolated from the immunized animal and screened by methods well known in the art to isolate those antibodies that specifically bind a peptide spanning amino acids 4-20 of both the acylated and des-acyl forms of hGhrelin. Methods for such isolation and screening are well known in the art. Such antibodies isolated by this process may further be altered to a chimeric or humanized form using methods well known in the art. Monoclonal anti-hGhrelin antibodies isolated by this process are contemplated to fall within the scope of the invention.

In a preferred embodiment, the invention provides anti-hGhrelin monoclonal antibodies resulting from the process described that preferably bind a human ghrelin peptide with an equilibrium dissociation constant, K_{D}, of 10⁻⁷ M or less, or 10⁻⁸ M or less, and more preferably 10⁻⁹ M or less (as determined by solid phase BIAcore® surface plasmon resonance at room temperature) and has the capacity to antagonize an activity of human ghrelin.

Anti-hGhrelin monoclonal antibodies of the invention inhibit a hGhrelin-mediated activity as represented, e.g., by the FLIPR assay described in Example 4 herein. Preferably, said hGhrelin-mediated activity is inhibited with an IC₅₀ of 40 nM or less, more preferably 10 nM or less, 5 nM or less, 4 nM or less, 3nM or less, most preferably 2 nM or less, or 1 nM or less or an IC₅₀ of 0.8 nM or less.

A preferred anti-hGhrelin Fab of the present invention comprises the amino acid sequence as shown in SEQ ID NOs: 3 and 10; that Fab is referred to herein as 1181. Exemplary polynucleotide sequences encoding the LCVR and HCVR of Fab 1181 are shown in SEQ ID NO: 1 and SEQ ID NO: 10 respectively.

In another embodiment, a preferred anti-hGhrelin Fab is that referred to herein as 1621. The 1621 Fab has a LCVR and a HCVR comprising a peptide with a sequence as shown in SEQ ID NO: 4 and SEQ ID NO: 13 respectively. Exemplary polynucleotide sequences encoding the LCVR and HCVR of 1621 are shown in SEQ ID NO: 2 and SEQ ID NO: 11 respectively. (See Tables 9-16 herein for all sequences and their locations in the Fabs).

A preferred anti-hGhrelin Fab of the present invention comprises the amino acid sequence as shown in SEQ ID NOs: 30 and 32; that Fab is referred to herein as 4641. Exemplary polynucleotide sequences encoding the LCVR and HCVR of Fab 4641 are shown in SEQ ID NO: 36 and SEQ ID NO: 37 respectively.

A preferred anti-hGhrelin Fab of the present invention comprises the amino acid sequence as shown in SEQ ID NOs: 31 and 33; that Fab is referred to herein as 1451. Exemplary polynucleotide sequences encoding the LCVR and HCVR of Fab 1451 are shown in SEQ ID NO: 34 and SEQ ID NO: 35 respectively.

The present invention is also directed to cell lines that produce an anti-hGhrelin monoclonal antibody (or fragment thereof) of the invention. Creation and isolation of cell lines producing a monoclonal antibody of the invention can be accomplished using routine screening techniques known in the art. Preferred cell lines include COS, CHO, SP2/0, NSO, HeLa and yeast (available from public repositories such as ATCC, American Type Culture Collection, Manassas, Va.).

A wide variety of host expression systems can be used to express an antibody of the present invention including prokaryotic (bacterial) and eukaryotic expression systems (such as yeast, baculoviral, plant, mammalian and other animal cells, transgenic animals, and hybridoma cells), as well as phage display expression systems. An example of a suitable bacterial expression vector is pUC119 and a suitable eukaryotic expression vector is a modified pcDNA3.1 vector with a weakened DHFR selection system. Other antibody expression systems are also known in the art and are contemplated herein.

An antibody of the invention can be prepared by recombinant expression of immunoglobulin light and heavy chain genes in a host cell. To express an antibody recombinantly, a host cell is transfected (or transformed, infected, or the like) with one or more recombinant expression vectors carrying DNA fragments encoding the immunoglobulin light and heavy chains of an antibody of the invention such that the light and/or heavy chains are expressed in the host cell. Preferably, the recombinant antibodies (or fraction thereof) are secreted into the medium in which the host cells are cultured, from which the antibodies can be recovered or purified. Standard recombinant DNA methodologies are used to obtain antibody heavy and light chain genes, incorporate these genes into recombinant expression vectors, and introduce the vectors into host cells. Such standard recombinant DNA technologies are described, for example, in Sambrook, Fritsch, and Maniatis (Eds.), Molecular Cloning; A Laboratory Manual, Second Edition, Cold Spring Harbor, N.Y., (1989); Ausubel, et al (Eds.) Current Protocols in Molecular Biology, Greene Publishing Associates, (1989); and in U.S. Pat. No. 4,816,397.

An isolated DNA encoding a HCVR region can be converted to a full-length heavy chain gene by operably linking the HCVR-encoding DNA to another DNA molecule encoding heavy chain constant regions (CH1, CH2, and CH3). The sequences of human heavy chain constant region genes are known in the art. See, e.g., Kabat, et al., Sequences of Proteins of Immunological Interest, Fifth Edition, U.S. Department of Health and Human Services, NIH Publication No. 91-3242 (1991). DNA fragments encompassing these regions can be obtained by standard PCR amplification. The heavy chain constant region can be of any type, (e.g., IgG, IgA, IgE, IgM or IgD), class (e.g., IgG₁, IgG₂, IgG₃ and IgG₄) or subclass constant region and any allotypic variant thereof as described in Kabat (supra), but most preferably is an IgG₄ or an IgG₁ constant region. Alternatively, the antigen binding portion can be a Fab fragment, Fab′ fragment, F(ab′)₂ fragment, Fd, or a single chain Fv fragment (scFv). For a Fab fragment heavy chain gene, the HCVR-encoding DNA can be operably linked to another DNA molecule encoding only a heavy chain CH1 constant region.

An isolated DNA encoding a LCVR region can be converted to a full-length light chain gene (as well as a Fab light chain gene) by operably linking the LCVR-encoding DNA to another DNA molecule encoding a light chain constant region, CL. The sequences of human light chain constant region genes are known in the art. See, e.g., Kabat, supra. DNA fragments encompassing these regions can be obtained by standard PCR amplification. The light chain constant region can be a kappa or lambda constant region.

To create an scFv gene, the HCVR- and LCVR-encoding DNA fragments are operably linked to another fragment encoding a flexible linker, e.g., encoding the amino acid sequence (Gly₄-Ser)₃, such that the HCVR and LCVR sequences can be expressed as a contiguous single-chain protein, with the LCVR and HCVR regions joined by the flexible linker. See, e.g., Bird, et al., Science 242:423-6 (1988); Huston, et al., Proc. Natl. Acad. Sci. USA 85:5879-83 (1988); McCafferty, et al., Nature 348:552-4 (1990).

To express an antibody of the invention, a DNA comprising a partial or full-length light and/or heavy chain, obtained as described above, is inserted into an expression vector such that the gene is operably linked to transcriptional and translational control sequences. The partial or full-length light and heavy chains may each be operably linked to a separate promoter sequence or they may be operably linked to one promoter. If the sequences comprising LCVR and HCVR (said sequence may further be operably linked to the constant region of the antibody) are present in the same vector and transcribed from one promoter to which they are both operably linked, a sequence comprising LCVR may be 5′ or 3′ to a sequence comprising HCVR. Furthermore, the LCVR and HCVR coding region in the vector may be separated by a linker sequence of any size or content, preferably such linker, when present, comprises a sequence encoding an internal ribosome entry site.

The expression vector and expression control sequences are chosen to be compatible with the expression host cell used. The antibody light chain gene and the antibody heavy chain gene can be inserted into separate expression vectors or, more typically, both genes are inserted into the same expression vector. The antibody genes are inserted into the expression vector by standard methods. Additionally, the recombinant expression vector can encode a signal peptide that facilitates secretion of the anti-ghrelin monoclonal antibody light and/or heavy chain from a host cell. The anti-ghrelin monoclonal antibody light and/or heavy chain gene can be cloned into the vector such that the signal peptide is operably linked in-frame to the amino terminus of the antibody chain gene. The signal peptide can be an immunoglobulin signal peptide or a heterologous signal peptide.

In addition to the antibody heavy and/or light chain gene(s), a recombinant expression vector of the invention carries regulatory sequences that control the expression of the antibody chain gene(s) in a host cell. The term “regulatory sequence” is intended to include promoters, enhancers and other expression control elements (e.g. polyadenylation signals), as needed, that control the transcription or translation of the antibody chain gene(s). The design of the expression vector, including the selection of regulatory sequences may depend on such factors as the choice of the host cell to be transformed, the level of expression of protein desired. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma virus.

In addition to the antibody heavy and/or light chain genes and regulatory sequences, the recombinant expression vectors of the invention may carry additional sequences, such as sequences that regulate replication of the vector in host cells (e.g., origins of replication) and one or more selectable marker genes. The selectable marker gene facilitates selection of host cells into which the vector has been introduced. For example, typically the selectable marker gene confers resistance to drugs, such as G418, hygromycin, or methotrexate, on a host cell into which the vector has been introduced. Preferred selectable marker genes include the dihydrofolate reductase (DHFR) gene (for use in DHFR-minus host cells with methotrexate selection/amplification), the neo gene (for G418 selection), and glutamine synthetase (GS) in a GS-negative cell line (such as NSO) for selection/amplification.

For expression of the light and/or heavy chains, the expression vector(s) encoding the heavy and/or light chains is transfected into a host cell by standard techniques e.g., electroporation, calcium phosphate precipitation, DEAE-dextran transfection and the like. Although it is theoretically possible to express the antibodies of the invention in either prokaryotic or eukaryotic host cells, preferably eukaryotic cells, and most preferably mammalian host cells, because such cells, are more likely to assemble and secrete a properly folded and immunologically active antibody. Preferred mammalian host cells for expressing the recombinant antibodies of the invention include Chinese Hamster Ovary (CHO cells) (including DHFR-CHO cells, described in Urlaub and Chasin, Proc. Natl. Acad. Sci. USA 77:4216-20 (1980), used with a DHFR selectable marker, e.g. as described in Kaufman and Sharp, J. Mol. Biol. 159:601-21 (1982)), NSO myeloma cells, COS cells, HeLa cells and SP2/0 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the host cell and/or the culture medium using standard purification methods.

Host cells can also be used to produce portions, or fragments, of intact antibodies, e.g., Fab fragments or scFv molecules. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all the DNA encoding either or both of the light and heavy chains that is not necessary for binding to ghrelin. The molecules expressed from such truncated DNA molecules are also encompassed by the antibodies of the invention.

In a preferred system for recombinant expression of an antibody of the invention, a recombinant expression vector encoding both the antibody heavy chain and the antibody light chain is introduced into DHFR-CHO cells by calcium phosphate-mediated transfection. Within the recombinant expression vector, the antibody heavy and light chain genes are each operably linked to separate enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Antibodies, or antigen-binding portions thereof, of the invention can be expressed in an animal (e.g., a mouse) that is transgenic for human immunoglobulin genes (see, e.g., Taylor, et al., Nucleic Acids Res. 20:6287-95(1992)). Plant cells can also be modified to create transgenic plants that express the antibody, or an antigen-binding portion thereof, of the invention.

The invention also provides recombinant expression vectors encoding both an antibody heavy chain and/or an antibody light chain. For example, in one embodiment, the invention provides a recombinant expression vector encoding:
a) an antibody heavy chain having a variable region comprising at least one peptide with an amino acid sequence selected from the group consisting of SEQ ID NOs: 14, 15, 16, 17, 18, 24, 25, 26 and 27; and further comprising a polynucleotide sequence encoding
b) an antibody light chain having a variable region comprising at least one peptide with an amino acid sequence selected from the group consisting of SEQ ID NOs: 5, 6, 7, 8, 9, 20, 21, 22, 23, 24, 28 and 29.

The invention also provides host cells into which one or more of the recombinant expression vectors of the invention have been introduced. Preferably, the host cell is a mammalian host cell, more preferably the host cell is a CHO cell, an NSO cell, a SP2/0 cell, a COS cell. Such cells are available from biological repositories such as the ATCC in Manassas, Va. Still farther the invention provides a method of synthesizing an antibody of the invention by culturing a host cell of the invention in a suitable culture medium until said antibody of the invention is synthesized. The method can further comprise isolating the antibody from the culture medium.

Once expressed, the intact antibodies, their dimers, individual light and heavy chains, or other immunoglobulin forms of the present invention can be purified according to standard procedures of the art, including ammonium sulfate precipitation, ion exchange, affinity, reverse phase, hydrophobic interaction column chromatography, gel electrophoresis and the like. Substantially pure immunoglobulins of at least about 90%, 92%, 94% or 96% homogeneity are preferred, and 98 to 99% or more homogeneity most preferred, for pharmaceutical uses. Once purified, partially or to homogeneity as desired, the peptides may then be used therapeutically or prophylactically, as directed herein.

Chimeric Antibodies

As used herein, the term “chimeric antibody” includes monovalent, divalent or polyvalent immunoglobulins. A monovalent chimeric antibody is a dimer formed by a chimeric heavy chain associated through disulfide bridges with a chimeric light chain. A divalent chimeric antibody is a tetramer formed by two heavy chain-light chain dimers associated through at least one disulfide bridge.

A chimeric heavy chain comprises an antigen-binding region derived from the heavy chain of a non-human antibody specific for ghrelin, which is linked to at least a portion of a human heavy chain constant region, such as CH1 or CH2.

A chimeric light chain comprises an antigen binding region derived from the light chain of a non-human antibody specific for ghrelin, linked to at least a portion of a human light chain constant region (CL).

Antibodies, fragments or derivatives having chimeric heavy chains and light chains of the same or different variable region binding specificity, can also be prepared by appropriate association of the individual polypeptide chains, according to known method steps.

With this approach, hosts expressing chimeric heavy chains are separately cultured from hosts expressing chimeric light chains, and the immunoglobulin chains are separately recovered and then associated. Alternatively, the hosts can be co-cultured and the chains allowed to associate spontaneously in the culture medium, followed by recovery of the assembled immunoglobulin or fragment.

Methods for producing chimeric antibodies are known in the art (see, e.g., U.S. Pat. Nos. 6,284,471; 5,807,715; 4,816,567; and 4,816,397).

In a preferred embodiment, a gene is created which comprises a first DNA segment that encodes at least the antigen-binding region of non-human origin (e.g., that of Fab 1181 or Fab 1621), such as functionally rearranged variable (V) region with joining (J) segment, linked to a second DNA segment encoding at least a part of a human constant (C) region as described un U.S. Pat. No. 6,284,471 (incorporated herein in its entirety).

Humanized Antibodies

A “humanized antibody” has CDRs that originate from a non-human (preferably a mouse monoclonal antibody) while framework and constant region, to the extent it is present, (or a substantial portion thereof, i.e., at least about 90%, 92%, 94%, 96%, 98% or 99%) are encoded by nucleic acid sequence information that occurs in the human genmline immunoglobulin region or in recombined or mutated forms thereof whether or not said antibodies are produced in human cells. Amino acids of the antibody may be altered to deimmunize the antibody, i.e., to minimize the chance that the body develops an allergic response to the antibody. A humanized antibody may be an intact antibody, a substantially intact antibody, a portion of an antibody comprising an antigen-binding site, or a portion of an antibody comprising a Fab fragment, Fab′ fragment, F(ab′)₂, or a single chain Fv fragment. It is contemplated that in the process of creating a humanized antibody, the amino acid at either termini of a CDR (see e.g., SEQ ID NOs: 5, 6, 7, 8, 9, 14, 15, 16, 17, 18, 20, 21, 22, 23, 24, 25, 26, 27, 28 or 29) maybe substituted with an amino acid that occurs in the human germline for that segment of adjoining framework sequence. Preferably a therapeutic antibody of the invention would have sequence of the framework and/or constant region derived from the mammal in which it would be used as a therapeutic so as to decrease the possibility that the mammal would illicit an immune response against the therapeutic antibody.

Humanized antibodies may be subjected to in vitro mutagenesis using methods of routine use in the art (or, when an animal transgenic for human Ig sequences is used, in vivo somatic mutagenesis) and, thus, the framework region amino acid sequences of the HCVR and LCVR regions of the humanized recombinant antibodies are sequences that, while derived from those related to human germline HCVR and LCVR sequences, may not naturally exist within the human antibody germline repertoire in vivo. It is contemplated that such amino acid sequences of the HCVR and LCVR framework regions of the humanized recombinant antibodies are at least 90%, 92%, 94%, 96%, 98% or most preferably at least 99% identical to a human gemline sequence.

Humanized antibodies have at least three potential advantages over non-human and chimeric antibodies for use in human therapy: (i) the effector portion is human, it may interact better with the other parts of the human immune system (e.g., destroy the target cells more efficiently by complement-dependent cytotoxicity or antibody-dependent cellular cytotoxicity); (ii) the human immune system should not recognize the framework or constant region of the humanized antibody as foreign, and therefore the antibody response against such an injected antibody should be less than that against a totally foreign non-human antibody or a partially foreign chimeric antibody; and (iii) injected non-human antibodies have been reported to have a half-life in the human circulation much shorter than the half-life of human antibodies. Injected humanized antibodies may have a half-life much like that of naturally occurring human antibodies, thereby allowing smaller and less frequent doses to be given.

Humanization may in some instances adversely affect antigen binding of the antibody. Preferably a humanized anti-hGhrelin monoclonal antibody of the present invention will possess a binding affinity for hGhrelin of not less than about 50%, more preferably not less than about 30%, and most preferably not less than about 5% of the binding affinity of the parent murine antibody, preferably Fab 1181, Fab 1621, Fab 1451 or Fab 4641, for hGhrelin. Preferably, a humanized antibody of the present invention will bind the same epitope as does Fab 1181, Fab 1621, Fab 1451 or Fab 4641 described herein. Said antibody can be identified based on its ability to compete with Fab 1181, Fab 1621, Fab 1451 or Fab 4641 for binding to acylated hGhrelin or des-acyl hGhrelin or to cells expressing acylated hGhrelin or des-acyl hGhrelin.

The design of humanized antibodies of the invention may be carried out as follows. In general, the humanized antibodies are produced by obtaining nucleic acid sequences encoding the HCVR and LCVR of an antibody which binds a hGhrelin epitope localized between amino acids 420 of hGhrelin, identifying the CDRs in said HCVR and LCVR (nonhuman), and grafting such CDR-encoding nucleic acid sequences onto selected human framework-encoding nucleic acid sequences. Preferably, the human framework amino acid sequences are selected such that the resulting antibody is likely to be suitable for in vivo administration in humans. This can be determined, e.g., based on previous usage of antibodies containing such human framework sequence. Preferably, the human framework sequence will not itself be significantly immunogenic.

Alternatively, the amino acid sequences of the frameworks for the antibody to be humanized (e.g., Fab 1181) will be compared to those of known human framework sequences the human framework sequences to be used for CDR-grafting will be selected based on their comprising sequences highly similar to those of the parent antibody, e.g., a murine antibody which binds hGhrelin. Numerous human framework sequences have been isolated and their sequences reported in the art. This enhances the likelihood that the resultant CDR-grafted humanized antibody, which contains CDRs of the parent (e.g., murine) antibody grafted onto selected human frameworks (and possibly also the human constant region) will substantially retain the antigen binding structure and thus retain the binding affinity of the parent antibody. To retain a significant degree of antigen binding affinity, the selected human framework regions will preferably be those that are expected to be suitable for in vivo administration, i.e., not immunogenic.

In either method, the DNA sequence encoding the HCVR and LCVR regions of the preferably murine anti-hGhrelin antibody are obtained. Methods for cloning nucleic acid sequences encoding immunoglobulins are well known in the art. Such methods may, for example, involve the amplification of the immunoglobulin-encoding sequences to be cloned using appropriate primers by polymerase chain reaction (PCR). Primers suitable for amplifying immunoglobulin nucleic acid sequences, and specifically murine HCVR and LCVR sequences have been reported in the literature. After such immunoglobulin-encoding sequences have been cloned, they will be sequences by methods well known in the art.

Once the DNA sequences encoding the CDRs and frameworks of the antibody which is to be humanized have been identified, (see e.g., Tables 9-16 herein), the amino acid sequences encoding the CDRs are then identified (deduced based on the nucleic acid sequences and the genetic code and by comparison to previous antibody sequences) and the CDR-encoding nucleic acid sequences are grafted onto selected human framework-encoding sequences. This may be accomplished by use of appropriate primers and linkers. Methods for selecting suitable primers and linkers for ligation of desired nucleic acid sequences is well within the ability of one of ordinary skill in the art.

After the CDR-encoding sequences are grafted onto the selected human framework encoding sequences, the resultant DNA sequences encoding the “humanized” variable heavy and variable light sequences are then expressed to produce a humanized Fv or humanized antibody which binds acylated and des-acyl hGhrelin. Typically, the humanized HCVR and LCVR are expressed as part of a whole anti-hGhrelin antibody molecule, i.e., as a fusion protein with human constant domain sequences whose encoding DNA sequences have been obtained from a commercially available library or which have been obtained using, e.g., one of the above described methods for obtaining DNA sequences, or are in the art. However, the HCVR and LCVR sequences can also be expressed in the absence of constant sequences to produce a humanized anti-hGhrelin Fv. Nevertheless, fusion of human constant sequences is potentially desirable because the resultant humanized anti-hGhrelin antibody may possess human effector functions.

Methods for synthesizing DNA encoding a protein of known sequence are well known in the art. Using such methods, DNA sequences which encode the subject humanized HCVR and LCVR sequences (with or without constant regions) are synthesized, and then expressed in a vector system suitable for expression of recombinant antibodies. This may be effected in any vector system which provides for the subject humanized HCVR and LCVR sequences to be expressed as a fusion protein with human constant domain sequences and to associate to produce functional (antigen binding) antibodies or antibody fragments.

Human constant domain sequences are well known in the art and have been reported in the literature. Preferred human constant light chain sequences include the kappa and lambda constant light chain sequences. Preferred human constant heavy chain sequences include human gamma 1, human gamma 2, human gamma 3, human gamma r, and mutated versions thereof which provide for altered effect or function, e.g. enhanced in vivo half-life, reduced Fc receptor binding, and the like.

In some instances, humanized antibodies produced by grafting CDRs (from an antibody which binds hGhrelin) onto selected human frameworks may provide humanized antibodies having the desired affinity to hGhrelin. However, it may be necessary or desirable to further modify specific residues of the selected human framework in order to enhance antigen binding. Preferably, those framework residues of the parent (e.g., murine) antibody which maintain or affect combining-site structures will be retained. These residues may be identified by X-ray crystallography of the parent antibody or Fab fragment, thereby identifying the three-dimensional structure of the antigen-binding site.

References further describing methods involved in humanizing a mouse antibody that maybe used are e.g., Queen et al., Proc. Natl. Acad. Sci. USA 88:2869, 1991; U.S. Pat. No. 5,693,761; U.S. Pat. No. 4,816,397; U.S. Pat. No. 5,225,539; computer programs ABMOD and ENCAD as described in Levitt, M., J. Mol. Biol. 168:595-620, 1983.

The present invention further embraces variants and the equivalents that are substantially homologous to the humanized antibodies and antibody fragments set forth herein. These are contemplated to contain 1 or 2 conservative substitution mutations within the CDRs of the antibody. For example, conservative substitution refers to the substitution of an amino acid with another within the same general class, e.g. one acidic amino acid with another acidic amino acid, one basic amino acid with another basic amino acid, or one neutral amino acid by another neutral amino acid. What is intended by a conservative amino acid substitution is well known in the art. These variants and equivalents substantially homologous to the humanized antibodies are also contemplated to contain a deletion of a terminal amino acid of a CDR.

Diagnostic Use

An antibody of the invention may be used to diagnose a disorder or disease associated with the expression of human ghrelin, i.e., either acylated or des-acyl form of ghrelin. In a similar manner, the antibody of the invention can be used in an assay to monitor ghrelin levels in a subject being treated for a ghrelin associated condition. Diagnostic assays include methods that utilize the antibody of the invention and a label to detect acylated ghrelin and/or des-acyl ghrelin in a sample, e.g., in a human body fluid or in a cell or tissue extract. Binding compositions, such as, e.g., antibodies, are used with or without modification, and are labeled by covalent or non-covalent attachment of a reporter molecule.

A variety of conventional protocols for measuring ghrelin, including ELISAs, RIAs, and FACS, are known in the art and provide a basis for diagnosing altered or abnormal levels of ghrelin expression. Normal or standard expression values are established using any art known technique, e.g., by combining a sample comprising a ghrelin polypeptide with, e.g., antibodies under conditions suitable to form a ghrelin:antibody complex. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, betagalactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of a radioactive material include ¹²⁵I, ¹³¹I, ³⁵S, or ³H. (See, e.g., Zola, Monoclonal Antibodies: A Manual of Techniques, CRC Press, Inc. (1987)).

The amount of a standard complex formed is quantitated by various methods, such as, e.g., photometric means. Amounts of ghrelin polypeptide expressed in subject, control, and samples (e.g., from biopsied tissue) are then compared with the standard values. Deviation between standard and subject values establishes parameters for correlating a particular disorder, state, condition, syndrome, or disease with a certain level of expression (or lack thereof) for a ghrelin polypeptide.

Once the presence of a disorder, state, condition, syndrome, or disease is established and a treatment protocol is initiated, assays are repeated on a regular basis to monitor the level of ghrelin expression. The results obtained from successive assays are used to show the efficacy of treatment over a period ranging from several days to months. With respect to disorders of cell proliferation (e.g., a cancer), the presence of an abnormal amount of ghrelin (either under- or over expressed) in biopsied tissue or fluid from a subject may indicate a predisposition for the development of a disorder, state, condition, syndrome, or disease of cell proliferation or it may provide a means for detecting such a disorder, state, condition, syndrome, or disease prior to the appearance of actual clinical symptoms. A more definitive initial detection may allow earlier treatment thereby preventing and/or ameliorating further progression of cell proliferation.

Pharmaceutical Composition

An antibody of the invention can be incorporated into pharmaceutical compositions suitable for administration to a subject. The compounds of the invention may be administered alone or in combination with a pharmaceutically acceptable carrier, diluent, and/or excipients, in single or multiple doses. The pharmaceutical compositions for administration are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate. Said compositions are designed in accordance with conventional techniques as in e.g., Remington, The Science and Practice of Pharmacy, 19^{th} Edition, Gennaro, Ed., Mack Publishing Co., Easton, Pa. 1995 which provides a compendium of formulation techniques as are generally known to practitioners.

A pharmaceutical composition comprising an anti-hGhrelin monoclonal antibody of the present invention can be administered to a subject at risk for or exhibiting pathologies associated with obesity or related disorders as described herein using standard administration techniques including oral, intravenous, intraperitoneal, subcutaneous, pulmonary, transdermal, intramuscular, intranasal, buccal, sublingual, or suppository administration.

A pharmaceutical composition of the invention preferably is a “therapeutically effective amount” or a “prophylactically effective amount” of an antibody of the invention. A “therapeutically effective amount” refers to an amount effective, at dosages and for periods of time necessary, to achieve the desired therapeutic result. A therapeutically effective amount of the antibody may vary according to factors such as the disease state, age, sex, and weight of the individual, and the ability of the antibody or antibody portion to elicit a desired response in the individual. A therapeutically effective amount is also one in which any toxic or detrimental effect of the antibody, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A therapeutically-effective amount is at least the minimal dose, but less than a toxic dose, of an active agent which is necessary to impart therapeutic benefit to a subject. Stated another way, a therapeutically-effective amount is an amount which induces, ameliorates or otherwise causes an improvement in the obese state of the mammal, e.g., by decreasing body mass index (BMI).

The route of administration of an antibody of the present invention may be oral, parenteral, by inhalation, or topical. Preferably, the antibodies of the invention can be incorporated into a pharmaceutical composition suitable for parenteral administration. The term parenteral as used herein includes intravenous, intramuscular, subcutaneous, rectal, vaginal, or intraperitoneal administration. Peripheral systemic delivery by intravenous or intraperitoneal or subcutaneous injection is preferred. Suitable vehicles for such injections are straightforward.

The pharmaceutical composition typically must be sterile and stable under the conditions of manufacture and storage in the container provided, including e.g. a sealed vial or syringe. Therefore, pharmaceutical compositions may be sterile filtered after making the formulation, or otherwise made microbiologically acceptable. A typical composition for intravenous infusion could have a volume as much as 250-1000 ml of fluid, such as sterile Ringer's solution, physiological saline, dextrose solution and Hank's solution and a therapeutically effective dose, (e.g., 1 to 100 mg/mL, or more) of antibody concentration. Therapeutic agents of the invention may be frozen or lyophilized for storage and reconstituted in a suitable sterile carrier prior to use. Lyophilization and reconstitution can lead to varying degrees of antibody activity loss (e.g. with conventional immunoglobulins, IgM antibodies tend to have greater activity loss than IgG antibodies). Dosages may have to be adjusted to compensate. Generally, pH between 6 and 8 is preferred.

As is well known in the medical arts, dosages for any one subject depends upon many factors, including the patient's size, body surface area, age, the particular compound to be administered, sex, time and route of administration, general health, and other drugs being administered concurrently. A typical dose can be, for example, in the range of 0.001 to 1000 μg; however, doses below or above this exemplary range are envisioned, especially considering the aforementioned factors. The daily parenteral dosage regimen is about 0.1 μg/kg to about 100 mg/kg of total body weight, preferably from about 0.3 μg/kg to about 10 mg/kg and more preferably from about 1 μg/kg to 1 mg/kg, even more preferably from about 0.5 to 10 mg/kg body weight per day. Progress may be monitored by periodic assessment.

Therapeutic Use

Ghrelin plays a role in the pathophysiology of obesity and a number of related disorders or diseases. Ghrelin is the first circulating hormone shown to stimulate feeding in humans following systemic administration. One study demonstrated that obese subjects do not demonstrate the decline in plasma ghrelin levels as seen after a meal in lean subjects and may therefore lead to increased food consumption (English, P. et al., J. Clin. End. & Metabolism, 87:2984-2987, 2002). Therefore, a pharmaceutical composition comprising an anti-hGhrelin monoclonal antibody of the invention may be used to treat or prevent obesity and/or obesity-related disorders such as NIDDM, Prader-Willi syndrome, impaired satiety, hyperphagia.

Obesity, also called corpulence or fatness, is the excessive accumulation of body fat, usually caused by the consumption of more calories than the body uses. The excess calories are then stored as fat, or adipose tissue. To be overweight, if moderate, is not necessarily to be obese, e.g., in muscular individuals. In general, however, a body weight of a subject that is 20 percent or more over the optimum tends to be associated with obesity. Alternatively, obesity may be defined in terms of Body Mass Index (BMI). Human BMI is defined as the body weight of a human in kilograms divided by the square of the height of that individual in meters. Typically, persons with a BMI of between 25 and 29 are considered overweight and a BMI of 29 or greater is considered obese. This may vary in some persons due to differences in gender or body frame. However, typically BMI of 25 or greater defines the point where the risk of disease increases due to excess weight. Assays for measuring energy expenditure, body composition and weight loss in animals that would be useful for determining effect of an antibody of the invention on an obese subject are known in the art, see e.g., International Patent Publication Number WO 01/87335 (incorporated herein by reference).

Hunger is a desire for food and is normal. Hunger typically occurs when caloric intake is less than caloric expenditure (negative energy balance) and in anticipation of an entrained meal even when the individual is in a positive energy balance. Hyperphagia and impaired satiety are defined as excessive ingestion of food beyond that needed for basic energy requirements. Ingestion may occupy unusual amounts of time. Eating may be obligatory and disrupt normal activity and can be symptomatic of various disorders. Hyperphagic or impaired satiety conditions may occur in association with central nervous system (CNS) disorders including gangliocytoma of the third ventricle, hypothalmic astrocytoma, Kleine-Levin Syndrome, Froehlich's Syndrome, Parkinson's Disease, genetic disorders including Praeder-Willi Syndrome (deletion on the long arm of chromosome 15), psychiatric disorders including anxiety, major depressive disorder, depressive phase of bipolar disorder, seasonal affective disorder, and schizophrenia, psychotropic medication, including delta-9 tetrahydrocannabinol, antidepressants and neuroleptics, may induce hyperphagia. Sleep disorders including sleep apnea is also associated with hyperphagia.

Type II diabetes mellitus, also called non-insulin dependent diabetes mellius (NIDDM), is present in subjects whose insulin their body is still capable of producing is not physiologically effective. An individual can be predisposed to NIDDM by both genetic and environmental factors. Heredity, obesity, and increased age play a major role in the onset of NIDDM. Risk factors include prolonged stress, sedentary lifestyle and certain medications affecting hormonal processes in the body. Eighty percent or more of the people with NIDDM are obese indicating obesity to be a predominant link to the development of NIDDM. An antibody of the invention may also be used to treat or prevent eating disorders including, but not limited to, bulimia, anorexia nervosa, binge eating and metabolic syndrome. An antibody of the invention may be used to treat or prevent cancer or cardiovascular disease.

The use of an anti-hGhrelin monoclonal antibody of the present invention for treating or preventing of at least one of the aforementioned disorders in which ghrelin activity is detrimental is also contemplated herein. Additionally, the use of an anti-ghrelin monoclonal antibody of the present invention for use in the manufacture of a medicament for the treatment of at least one of the aforementioned disorders in which ghrelin activity is detrimental is contemplated.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, includes administration of a compound of the present invention for treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, ie., causing regression of the disease or disorder or alleviating symptoms or complications thereof. Treatment may be in conjunction with behavior modification such as limitation of food intake and exercise. Treating obesity therefore includes inhibition of food intake, inhibition of weight gain, and/or inducing weight loss in subjects in need thereof. Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation.

The following examples are offered for illustrative purposes only, and are not intended to limit the scope of the present invention in any way;

### EXAMPLES

### Example 1

### Anti-Ghrelin Fab Synthesis

The CDR and framework sequences disclosed herein are identified from clones of Fab fragments isolated from antibody libraries generated from antibody RNA created by immunized C57B16 wild-type mice using Omniclonal™ antibody technology (Biosite®, San Diego, Calif.). The mice are immunized with human ghrelin acylated at the His residue at position 9 (SEQ ID NO: 19) and to which a C-terminal cysteine is added. To improve the immunogenicity of this peptide, keyhole limpet hemocyanin is conjugated to the peptide through a C-terminal cysteine according to standard methods. Sequences comprised within Fab 1181, Fab 1621, Fab 1451 and Fab 4641, isolated from this immunization are shown in Tables 9-16 herein.

### Example 2

### Competitive ELISA Assay

Anti-hGhrelin Fabs 1181 and 1621 are tested in a competitive ELISA assay, an assay in which a compound that might compete with an antigen for binding to an antibody is first combined with the antibody in solution phase. Then binding of the antibody to the antigen coated on a plate is measured.

Each well of a 96-well plate is coated with 60 μl BSA-hGhrelin antigen (ie., BSA conjugated, full-length, acylated human ghrelin, 2 μg/ml in carbonate buffer, pH 9.6). The plate was incubated at 4° C. overnight. The wells are aspirated and washed twice with washing buffer (20 mM Tris-Cl, pH 7.4, 0.15 M NaCl, 0.1% Tween 20). Compounds (i.e., human ghrelin or ghrelin analogs) are diluted into antibody solution. The antibody solution has a mouse anti-human ghrelin Fab. The compound concentration is varied as listed in Tables 2-5 below, but the Fab concentration is kept constant at 0.1 μg/ml in blocking solution (10 mg/ml BSA in wash buffer). After a 1-hour incubation at room temperature, 50 μl of compound-antibody solution is added to the BSA-hGhrelin coated wells in triplicate. The plates are incubated for 1 hour at room temperature. The wells are then washed three times with washing buffer.

Peroxidase-conjugated secondary antibody (50 μl goat anti-mouse kappa HRP (Southern Biotech), diluted 1:2000 in blocking buffer) is added to each well and incubated for 1 hour at room temperature. The wells are then washed 4 times with washing buffer. Fifty microliters of chromogenic substrate (i.e., OPD substrate) is added to each well and allowed to develop at room temperature for 10 minutes. The reaction is stopped by adding 100 μl 1N HCl to each well. The absorbance of the wells is read at 490 nm.

Six different compounds are tested at concentrations of 0.2 μg/ml, 1.0 μg/ml and 5 μg/ml using Fab 1181 and, separately, Fab 1621. The six compounds are: (1) Ghrelin (1-8 acyl), which is the first eight amino acids of human ghrelin acylated, via an ester linkage, with an octanoyl group on the serine at position 3; (2) Ghrelin (9-28), which is amino acids 9-28 of human ghrelin [not acylated]; (3) Ghrelin (1-8, desacyl), which is the first eight amino acids of human ghrelin not acylated; (4) hGhrelin which is full-length human ghrelin (1-28) acylated with an octanoyl group on the serine at position 3; (5) hGhrelin (desacyl) which is full-length human ghrelin not acylated; and (6) rGhrelin, which is full-length, acylated rat ghrelin. The average absorbance from triplicate wells is determined. These values are listed below in Tables 2 and 3.

These data demonstrate that Fab 1181 and Fab 1621 bind a similar epitope. Both Fabs bind full-length hGhrelin regardless of whether or not it is acylated, indicating that the acyl group at amino acid 3 of hGhrelin is not a part of the epitope. When rat ghrelin is tested, it competes with hGhrelin for binding the Fabs, but it competes poorly by comparison to when hGhrelin is the compound. Rat ghrelin is identical to human ghrelin except at amino acids 11 and 12. These data indicate that amino acids 11 and 12 are a part of the epitope to which Fabs 1181 and 1621 bind. Furthermore, hGhrelin (1-8 acyl) and hGhrelin (1-8 desacyl) do not compete with full-length hGhrelin for binding Fabs 1181 and 1621. There is no statistical competition seen with the hGhrelin (9-28) peptide with fill-length hGhrelin for binding Fabs 1181 and 1621. These data indicate that ghrelin polypeptides spanning amino acids 1-8 and 9-28 do not provide the complete epitope. However, ghrelin polypeptide spanning amino acids 1-28 of ghrelin does provide the complete antigenic epitope; therefore, the antigenic epitope spans the junction between amino acids 8 and 9. It is commonly believed in the art that a linear epitope has an optimal length of 8-12 amino acids and that the minimal size of a linear epitope is about 6 amino acid residues. However, a linear epitope may be greater than 30 amino acids in length (See e.g., Oleksiewicz, M B et al., J. Virology, 75:3277-3290, 2001; Torrez-Martinez, N., et al., Virology, 211: 336-338, 1995).

TABLE 2
Fab 1181

| | Compound | | | |
|---|---|---|---|---|
| Compound | Concentration | | Ave. OD | Std Dev. |
| Ghrelin (1-8 acyl) | 0 | | 1.2266 | 0.1307 |
| | 0.2 | μg/ml | 1.2609 | 0.0209 |
| | 1 | μg/ml | 1.2234 | 0.0524 |
| | 5 | μg/ml | 1.2139 | 0.0450 |
| Ghrelin (9-28) | 0 | | 1.2266 | 0.1307 |
| | 0.2 | μg/ml | 1.1313 | 0.0867 |
| | 1 | μg/ml | 1.1699 | 0.0495 |
| | 5 | μg/ml | 1.108 | 0.0456 |
| Ghrelin (1-8, | 0 | | 1.2266 | 0.1307 |
| desacyl) | 0.2 | μg/ml | 1.2157 | 0.045 |
| | 1 | μg/ml | 1.1919 | 0.0328 |
| | 5 | μg/ml | 1.1759 | 0.0264 |
| hGhrelin | 0 | | 1.2266 | 0.1307 |
| | 0.2 | μg/ml | 0.0784 | 0.0457 |
| | 1 | μg/ml | 0.0497 | 0.0057 |
| | 5 | μg/ml | 0.0445 | 0.0046 |
| hGhrelin (desacyl) | 0 | | 1.2266 | 0.1307 |
| | 0.2 | μg/ml | 0.1096 | 0.0332 |
| | 1 | μg/ml | 0.0573 | 0.0065 |
| | 5 | μg/ml | 0.0533 | 0.0045 |
| rGhrelin | 0 | | 1.2266 | 0.1307 |
| | 0.2 | μg/ml | 1.1205 | 0.0412 |
| | 1 | μg/ml | 0.8831 | 0.0449 |
| | 5 | μg/ml | 0.178 | 0.0077 |

TABLE 3
Fab 1621

| | Compound | | | |
|---|---|---|---|---|
| Compound | Concentration | | Ave. OD | Std. Dev. |
| Ghrelin (1-8 acyl) | 0 | | 1.1749 | 0.2294 |
| | 0.2 | μg/ml | 1.3602 | 0.0607 |
| | 1 | μg/ml | 1.3203 | 0.0399 |
| | 5 | μg/ml | 1.2186 | 0.0147 |
| Ghrelin (9-28) | 0 | | 1.1749 | 0.095 |
| | 0.2 | μg/ml | 1.0449 | 0.2735 |
| | 1 | μg/ml | 1.0497 | 0.2232 |
| | 5 | μg/ml | 1.0153 | 0.1613 |
| Ghrelin (1-8, | 0 | | 1.1749 | 0.095 |
| desacyl) | 0.2 | μg/ml | 1.3832 | 0.045 |
| | 1 | μg/ml | 1.3287 | 0.0328 |
| | 5 | μg/ml | 1.2603 | 0.0264 |
| hGhrelin | 0 | | 1.1749 | 0.095 |
| | 0.2 | μg/ml | 0.1314 | 0.0457 |
| | 1 | μg/ml | 0.0495 | 0.0057 |
| | 5 | μg/ml | 0.043 | 0.0046 |
| hGhrelin (desacyl) | 0 | | 1.1749 | 0.095 |
| | 0.2 | μg/ml | 0.1879 | 0.0332 |
| | 1 | μg/ml | 0.0621 | 0.0065 |
| | 5 | μg/ml | 0.0499 | 0.0045 |
| rGhrelin | 0 | | 1.1749 | 0.095 |
| | 0.2 | μg/ml | 0.9986 | 0.0412 |
| | 1 | μg/ml | 0.9323 | 0.0449 |
| | 5 | μg/ml | 0.4291 | 0.0077 |

### Example 3

### FLIPR in vitro Activity Assay

The in vitro FLIPR® Calcium Assay system Molecular Devices) is used with hamster AV12 cells stably transfected to express the GHS-R1a human ghrelin receptor. This assay evaluates changes in intracellular calcium as a means of detecting ghrelin/GHS-R1a binding and signaling in the presence or absence of a Fab of the invention. This functional assay is used to further map the location of the epitope to which the monoclonal antibodies of the invention bind.

AV12 cells are grown in growth media (DMEM/F12 (3:1), 5% fetal bovine serum, 50 μg/ml hygromycin and 50 μg/ml zeocin) to about 50-90×10⁶ cells per T-150 flask. The cells are then trypsinized, washed and distributed into Biocoat black poly-D-lysine coated plates (60,000 cells in 100 μl growth media per well). The cells are incubated for about 20 hours at 37° C. in 5% CO₂. The media is removed from the plate and 150 μl HBSS (Gibco 14025) is added to each well and then removed. Then dye is loaded into the cells by adding to each well 50 μl loading buffer [5 μM Fluo-4AM (Molecular Devices), 0.05% Pluronic in FLIPR buffer [Hank's Balanced Salt with calcium (HBSS, Gibco 14025-092) and 0.75% BSA (Gibco)]. The plate is Irther incubated at 37° C. in 5% CO₂ for one hour. The wells are then washed twice with HBSS and 50 μl FLIPR buffer is then added per well.

Samples are prepared by combining 7.2 μl calcium concentrate (CaCl₂-2H₂O in water at 3.7 mg/ml mixed 1:1 with HBSS and filter sterilized) with 30 μl peptide, 30 μl Fab (of varying concentration), and 16.8 μl hGhrelin (2.5 μM stock) in 3.75% BSA/50% HBSS. The final concentration of the sample solution is 0.75% BSA, and calcium at approximately the same concentration as in the FLIPR buffer. Fifty microliters of the sample solution is added to the 50 μl FLIPR buffer in the well with the AV12 cells. The final concentration of the peptide is 100 nM and the final concentration of the hGhrelin is 0.83 nM. The cell plate is shaken for 15 seconds prior to loading it into the FLIPR instrument. Test samples or control samples are added to each well, and read by a Fluorometric Imaging Plate Reader (Molecular Devices).

If there is no Fab or an irrelevant antibody present in the solution, the fill-length hGhrelin will be free to bind the GHS-R1a receptor on the AV12 cells and signal transduction will occur resulting in comparatively high values in the assay. If a Fab is present that binds to the full-length hGhrelin in the solution, then the full-length hGhrelin binding to the GHS-R1a receptor is inhibited and signal transduction is thereby inhibited resulting in comparatively lower values in the assay. However, if a peptide (i.e., a fragment of human ghrelin) is also added to the solution and the Fab binds the peptide, then the full-length hGhrelin is not prevented from binding the GHS-R1a receptor, signal transduction is not inhibited, and the values in the assay are comparatively high. Conversely, if a peptide is added to the solution and the Fab does not bind the peptide, then the Fab will be available to bind the full-length hGhrelin in the solution and the values in the assay will be comparatively low. Notably, the peptide fragments tested are not active and will not bind the GHS-R1a receptor; therefore, their presence will not contribute to background levels. The peptides competing with hGhrelin for Fab binding were used in the assay at a concentration over 50 times that of hGhrelin. The Fab concentration used was determined by titration to be a level that will give approximately 95% inhibition of 1 nM hghrelin activity.

The peptides (amino acid numbers correspond to those of human ghrelin as shown in SEQ ID NO: 20) tested and results obtained are listed in Table 4 below. Results stated are for both Fabs; standard deviation of the results was typically about 10-15% due to assay variation. Notably, human ghrelin peptide spanning amino acids 4-20 resulted in significant reduction of 1621 and 1181 Fab inhibition while peptide 7-16 had no reduction of inhibition of either Fab indicating that human ghrelin sequence beyond 7-16 was necessary for antibodies of the invention yet the epitope was located within the human ghrelin peptide spanning amino acids 4-20.

Fab 4641 (at 5 nM) and Fab 1451 (at 20 nM), when run in a similar assay with 100 nM peptide spanning 4-20 of human ghrelin, both resulted in a reduction of Fab inhibition. Fab 4641 (at 5 nM) and Fab 1451 (at 20 nM), when run in a similar assay with 100 nM peptide spanning 14-28 of human ghrelin, both resulted in no reduction of Fab inhibition. The results indicate that Fab 1451 and Fab 4641 are unable to bind a peptide spanning amino acids 14-28 of human ghrelin but bind a peptide spanning amino acids 4-20 of human ghrelin.

TABLE 4
Inhibition of hGhrelin at 0.83 nM

| Peptide | Fab 1621 | Fab 1181 | Result |
|---|---|---|---|
| 1-28 | 1.75 nM | 0.88 nM | 100% reduction Fab inhibition |
| des acyl | | | |
| 1-8 | 1.75 nM | 0.88 nM | No reduction Fab inhibition |
| des acyl | | | |
| 13-19 | 1.75 nM | 0.88 nM | No reduction Fab inhibition |
| 18-28 | 1.78 nM | 0.88 nM | No reduction Fab inhibition |
| 4-28 | 1.5 nM | 0.95 nM | 60% reduction Fab 1621 |
| | | | inhibition |
| | | | 80% reduction Fab 1181 |
| | | | inhibition |
| 9-28 | 1.5 nM | 0.74 nM | No reduction Fab inhibition |
| 7-16 | 1.95 nM | 1.1 nM | No reduction Fab inhibition |
| 4-20 | 1.5 nM | 0.95 nM | 52% reduction Fab 1621 |
| | | | inhibition |
| | | | 74% reduction Fab 1181 |
| | | | inhibition |
| 4-16 | 1.5 nM | 0.95 nM | 13% reduction Fab 1621 |
| | | | inhibition |
| | | | 30% reduction Fab 1181 |
| | | | inhibition |

### Example 4

### FLIPR Assay with Active Analogs

Active human ghrelin analogs or full-length, acylated rat ghrelin were combined with a Fab of the invention to determine if the Fab could inhibit the analog activity. This FLIPR Assay is performed substantially like that described in Example 3 herein, with the following exceptions. Analogs tested here (as shown in Table 5 below) are active and bind the GHS-1a receptor to which full-length acylated hGhrelin binds. Therefore, no full-length acylated hGhrelin is added to the sample in this assay.

The active analogs are used at a concentration that yields sub-maximal activity. The analogs are incubated with the Fab at concentrations known to fully inhibit 1 nM acylated hGhrelin.

Samples for this assay are prepared by combining 7.2 μl calcium concentrate (CaCl₂-2H₂O in water at 3.7 mg/ml mixed 1:1 with HBSS and filter sterilized) with 60 μl Fab (of varying concentration), and 16.8 μl peptide in 3.75% BSA/50% HBSS. The final concentration of the sample solution is 0.75% BSA, and calcium at approximately the same concentration as in the FLIPR buffer. Fifty microliters of the sample solution is added to the 50 μl FLIPR buffer in the well with the AV12 cells. The final concentration of the peptide and the Fab is stated in Table 5 below. Other aspects of the assay are the same as described in Example 3. Results are listed below.

TABLE 5

| Active Peptide | Fab 1621 | Fab 1181 | Result |
|---|---|---|---|
| Rat ghrelin | 70.1 nM | 17.7 nM | 36% reduction rat |
| (1 nM) | | | ghrelin activity |
| Human 1-13 | 175 nM | 132 nM | no inhibition of |
| acyl (1.5 nM) | | | activity |
| Human 1-8 | 175 nM | 132 nM | no inhibition of |
| acyl (11 nM) | | | activity |

TABLE 6
Inhibition of hGhrelin at 0.8 nM

| Fab | IC50 (nM) |
|---|---|
| 1181 | 0.80 |
| 1621 | 1.64 |

### Example 5

### Affinity Measurement of Monoclonal Antibodies

The affinity (K_{D}) of anti-ghrelin Fabs 1181 and 1621 are measured using a BIAcore® 2000 instrument containing a CM5 sensor chip. The BIAcore® utilizes the optical properties of surface plasmon resonance to detect alterations in protein concentration of interacting molecules within a dextran biosensor matrix. Except where noted, all reagents and materials are purchased from BIAcore® AB (Upsala, Sweden). All measurements are performed at 25° C. Samples containing rat or human ghrelin (full length, acylated) are dissolved in HBS-EP buffer (150 mM sodium chloride, 3 mM EDTA, 0.005% (w/v) surfactant P-20, and 10 mM HEPES, pH 7.4). A capture antibody, goat anti-mouse Kappa (Southern Biotechnology, Inc), is immobilized onto flow cells using amine-coupling chemistry. Flow cells (1-4) are activated for 7 minutes with a 1:1 mixture of 0.1 M N-hydroxysuccinimide and 0.1 M 3-(N,N-dimethylamino)propyl-N-ethylcarbodiimide at a flow rate of 10 μl/min. Goat anti-mouse Kappa (30 μg/mL in 10 mM sodium acetate, pH 4.5) is manually injected over all 4 flow cells at a flow rate of 10 μL/min. The surface density is monitored and additional goat anti-mouse Kappa is injected if needed to individual cell until all flow cells reach a surface density of 4500-5000 response units (RU). Surfaces are blocked with a 7 minute injection of 1 M ethanolamine-HCl, pH 8.5(10 μl/min). To ensure complete removal of any noncovalently bound goat anti-mouse Kappa, 15 μL of 10 mM glycine, pH 1.5 is injected twice. Running buffer used for kinetic experiments contained 10 mM HEPES, pH 7.4, 150 mM NaCl, 0.005% P20.

Collection of kinetic binding data is performed at maximum flow rate (100 μL/min) and a low surface density to minimize mass transport effects. Each analysis cycle consists of (i) capture of 300-350 RU of Fabs(BioSite) by injection of 5-10 μL of 5 μg/ml solution over flow cell 2, 3 and 4 for different Fabs at a flow rate of 10 μL/min., (ii) 200 μL injection (2 min) of hGhrelin (concentration range of 50 nM to 1.56 nM in 2-fold dilution increments) over all 4 flow cells with flow cell 1 as the reference flow cell, (iii) 10 min dissociation (buffer flow), (iv) regeneration of goat anti-mouse Kappa surface with a 15 sec injection of 10 mM glycine, pH 1.5, (v) a 30 sec blank injection of running buffer, and (vi) a 2 min stabilization time before start of next cycle. Signal is monitored as flow cell 2 minus flow cell 1, flow cell 3 minus flow cell I and flow cell 4 minus flow cell 1. Samples and a buffer blank are injected in duplicate in a random order. Data are processed using BIAevaluation 3.1 software and data are fit to a 1:1 binding model in CLAMP global analysis software. The average of three measurements is reported below for Fab 1181 with human ghrelin; one measurement is reported for Fab 1621 with human ghrelin, Fab 1181 with rat ghrelin, and Fab 1621 with rat ghrelin.

TABLE 7

| | |
|---|---|
| Acylated Human Ghrelin | Des-acyl Human Ghrelin |

| Fab | kₒₙ | Std Dev | Fab | kₒₙ |
|---|---|---|---|---|
| 1181 | 3.39 × 10⁶ | 8.99 × 10⁵ | 1181 | 7.02 × 10⁵ |
| 1621 | 3.61 × 10⁶ | 1.44 × 10⁶ | 1621 | 7.93 × 10⁵ |
| Fab | k_{off} | Std Dev | Fab | k_{off} |
| 1181 | 6.22 × 10⁻⁴ | 5.24 × 10⁻⁵ | 1181 | 3.04 × 10⁻⁴ |
| 1621 | 2.1 × 10⁻³ | 2.46 × 10⁻⁵ | 1621 | 1.38 × 10⁻³ |
| Fab | K_{D} (k_{off}/kₒₙ) (M) | Std Dev | Fab | K_{D} (M) |
| 1181 | 2.02 × 10⁻¹⁰ | 9.24 × 10⁻¹¹ | 1181 | 4.34 × 10⁻¹⁰ |
| 1621 | 6.80 × 10⁻¹⁰ | 3.13 × 10⁻¹⁰ | 1621 | 1.74 × 10⁻⁹ |

TABLE 8
Acylated Rat Ghrelin

| Fab | kₒₙ |
|---|---|
| 1181 | 2.02 × 10⁶ |
| 1621 | 1.53 × 10⁶ |
| Fab | k_{off} |
| 1181 | 2.11 × 10⁻² |
| 1621 | 8.73 × 10⁻³ |
| Fab | K_{D} (k_{off}/kₒₙ) (M) |
| 1181 | 1.04 × 10⁻⁸ |
| 1621 | 5.70 × 10⁻⁹ |

TABLE 9

Fab 1181 Light chain variable region DNA
and amino acid sequence.

```
D V V M T Q T P L S L P V S L 15
GATGTTGTGATGACCCAAACTCCACTCTCCCTGCCTGTCAGTCTT
                                                  CDR1
G D Q A S I S C R S S Q S L V 30
GGAGATCAAGCCTCCATCTCTTGCAGATCTAGTCAGAGCCTTGTA
H S N G N T Y L H W Y L Q K P 45
CACAGTAATGGAAACACCTATTTACATTGGTACCTGCAGAAGCCA
                                                            CDR2
G Q S P K L L I Y K V S N R F 60
GGCCAGTCTCCAAAGCTCCTGATCTACAAAGTTTCCAACCGATTT
S G V P D R F S G S G S G T D 75
TCTGGGGTCCCAGACAGGTTCAGTGGCAGTGGATCAGGGACAGAT
F T L K I S R V E A E D L G V 90
TTCACACTCAAGATCAGCAGAGTGGAGGCTGAGGATCTGGGAGTT
                                        CDR3
Y F C S Q S T H V P Y T F G G 105
TATTTCTGCTCTCAAAGTACACATGTTCCGTACACGTTCGGAGGG
G T K L E I K R 113
GGGACCAAGCTGGAAATAAAACGG
```

TABLE 10

Fab 1181 Heavy chain variable region DNA
and amino acid sequence

```
Q V Q L Q Q S G A E L V R S G 15
CAGGTCCAGCTGCAGCAGTCTGGGGCAGAGCTTGTGAGGTCAGGG
                                                                      CDR1
A S V K L S C T A S G F N I K 30
GCCTCAGTCAAGTTGTCCTGCACAGCTTCTGGCTTCAACATTAAA
D Y F M Q W V K Q R P E Q G L 45
GACTACTTTATGCAGTGGGTGAAGCAGAGGCCTGAACAGGGCCTG
                                        CDR2
E W I G W I D P E N G E T G Y 60
GAGTGGATTGGATGGATTGATCCTGAGAATGGTGAAACTGGATAT
A P K F Q G K A T M T A D T A 75
GCCCCGAAGTTCCAGGGCAAGGCCACTATGACTGCAGACACAGCC
S N T A Y L Q L S S L T S E D 90
TCCAATACAGCCTACCTGCAACTCAGCAGCCTGACATCTGAGGAC
                                                            CDR3
T A L Y Y C N A P S V V A K Y 105
ACTGCCCTGTATTACTGTAATGCACCTTCGGTCGTGGCTAAATAC
F D V W G A G T T V T V S S 119
TTCGATGTCTGGGGCGCAGGGACCACGGTCACCGTCTCCTCA
```

TABLE 11

Fab 1621 Light chain variable region DNA
and amino acid sequence

```
D V V L T Q T P L S L P V S L 15
GATGTTGTGCTGACCCAAACTCCACTCTCCCTGCCTGTCAGTCTT
                                                                      CDR1
G D Q A S I S C R S S Q S L V 30
GGAGATCAAGCCTCCATCTCTTGCAGATCTAGTCAGAGCCTTGTA
H S N G S T Y L H W Y L Q K P 45
CACAGTAATGGAAGCACCTATTTACATTGGTACCTGCAGAAGCCA
                                                            CDR2
G Q S P K L L I Y K V S N R F 60
GGCCAGTCTCCAAAGCTCCTGATCTACAAAGTTTCCAACCGATTT
S G V P D R F S G S G S G T D 75
TCTGGGGTCCCAGACAGGTTCAGTGGCAGTGGATCAGGGACAGAT
F T L K I S R V E A E D L G V 90
TTCACACTCAAGATCAGCAGAGTGGAGGCTGAGGATCTGGGAGTT
                              CDR3
Y F C S Q S T H V P Y T F G G 105
TATTTCTGCTCTCAAAGTACACATGTTCCGTACACGTTCGGAGGG
G T K L E I R R 113
GGGACCAAGCTGGAAATAAGACGG
```

TABLE 12

1621 Heavy chain variable region DNA
and amino acid sequence

```
Q V Q L Q Q S G A E L V R S G 15
CAGGTCCAGCTGCAGCAGTCTGGGGCAGAGCTTGTGAGGTCAGGG
                                                                      CDR1
A S V K L S C T A S G F N I K 30
GCCTCAGTCAAGTTGTCCTGCACAGCTTCTGGCTTCAACATTAAA
D Y F V Q W V K Q R P E Q G L 45
GACTACTTTGTGCAGTGGGTGAAGCAGAGGCCTGAACAGGGCCTG
                                                  CDR2
E W I G W I D P E N G E T G Y 60
GAGTGGATTGGATGGATTGATCCTGAGAATGGTGAAACTGGATAT
A P K F Q G K A T M T A D T A 75
GCCCCGAAGTTCCAGGGCAAGGCCACTATGACTGCAGACACAGCC
S N T A Y L Q L S S L T S E D 90
TCCAATACAGCCTACCTGCAACTCAGCAGCCTGACATCTGAGGAC
                                                            CDR3
T A L Y F C N A P S V V A K Y 105
ACTGCCCTGTATTTCTGTAATGCACCTTCGGTAGTGGCTAAATAC
F D V W G A G T T V T V S S 119
TTCGATGTCTGGGGCGCAGGGACCACGGTCACCGTCTCCTCA
```

TABLE 13

4641 Light chain variable region DNA
and amino acid sequence

```
D V V M T Q T P L S L P V S L 15
GATGTTGTGATGACCCAAACTCCACTCTCCCTGCCTGTCAGTCTT
G D Q A S I S C R S S Q S L V 30
GGAGATCAAGCCTCCATCTCTTGCAGATCTAGTCAGAGCCTTGTA
          CDR1
H S D G N T Y L H W Y L Q K P 45
CACAGTGATGGAAACACCTATTTACATTGGTACCTGCAGAAGCCA
                                                                      CDR2
G Q S P K L L I Y K V S N R F 60
GGCCAGTCTCCAAAGCTCCTGATCTACAAAGTTTCCAACCGATTT
S G V P D R F S G S G S G T D 75
TCTGGGGTCCCAGACAGGTTCAGTGGCAGTGGATCAGGGACAGAT
F T L K I S R V E A E D L G V 90
TTCACACTCAAGATCAGCAGAGTGGAGGCTGAGGATCTGGGAGTT
                            CDR3
Y F C S Q S T Y V S Y T F G G 105
TATTTCTGCTCTCAAAGTACATATGTTTCGTACACGTTCGGAGGG
G T K L E I K R A D A A P T V 120
GGGACCAAGCTGGAAATAAAACGGGCTGATGCTGCACCAACTGTA
```

TABLE 14

4641 Heavy chain variable region DNA
and amino acid sequence

```
Q V Q L Q Q S R P E L V R P G 15
CAGGTCCAGCTGCAGCAGTCTCGGCCTGAACTGGTGAGGCCTGGG
                                                                      CDR1
A S V K M S C R A S A Y T F T 30
GCTTCAGTGAAGATGTCCTGCAGGGCTTCAGCCTATACCTTCACC
T Y W M H W V R Q R P G Q G L 45
ACCTACTGGATGCACTGGGTGAGACAGAGGCCTGGACAAGGCCTT
                                                  CDR2
E W I G M I D P S N S D T W L 60
GAGTGGATTGGCATGATTGATCCTTCCAATAGTGATACTTGGTTA
N Q K F K D K A T L N V D R S 75
AATCAGAAGTTCAAGGACAAGGCCACATTGAATGTAGACAGATCC
S N T A Y M Q L T S L T S E D 90
TCCAACACAGCCTACATGCAGCTCACCAGCCTGACATCTGAGGAC
                                                  CDR3
S A V Y Y C A R T G F D Y W G 105
TCTGCAGTCTATTACTGTGCAAGAACTGGTTTTGACTACTGGGGC
Q G T T L T V S S 114
CAAGGCACCACTCTCACAGTCTCCTCA
```

TABLE 15

1451 Light chain variable region DNA
and amino acid sequence

```
D V V L T Q T P L 15
GATGTTGTGCTGACCCAAACTCCACTC
S L P V S L G D Q A S I S C R 30
TCCCTGCCTGTCAGTCTTGGAGATCAAGCCTCCATCTCTTGCAGA
          CDR1
S S Q S L V H S N G N T Y L H 45
TCTAGTCAGAGCCTTGTACACAGTAATGGAAACACCTATTTACAT
W Y L Q K P G Q S P K L L I Y 60
TGGTACCTGCAGAAGCCAGGCCAGTCTCCAAAGCTCCTGATCTAC
          CDR2
K V S N R F S G V P D R F S G 75
AAAGTTTCCAACCGATTTTCTGGGGTCCCAGACAGGTTCAGTGGC
S G S G T D F T L K I S R V E 90
AGTGGATCAGGGACAGATTTCACACTCAAGATCAGCAGAGTGGAG
CDR3
A E D L G V Y F C S Q S T H V 105
GCTGAGGATCTGGGAGTTTATTTCTGCTCTCAAAGTACACATGTT
P Y T F G G G T K L E I K R 119
CCGTACACGTTCGGTGGAGGCACCAAGCTGGAAATCAAACGG
```

TABLE 16

1451 Heavy chain variable region DNA
and amino acid sequence

```
Q V H V K Q S G P E L V R P G 15
CAGGTCCACGTGAAGCAGTCTGGGCCTGAACTGGTGAGGCCTGGG
                                                                      CDR1
A S V K M S C K A S A Y T F T 30
GCTTCAGTGAAGATGTCCTGCAAGGCTTCAGCCTATACCTTCACC
T Y W M H W V K Q R P G Q G L 45
ACCTACTGGATGCACTGGGTGAAACAGAGGCCTGGACAAGGCCTT
                                        CDR2
E W I G M I D P Y N S E T W L 60
GAGTGGATTGGCATGATTGATCCTTACAATAGTGAAACTTGGTTA
N Q K F K D K A T L N V D R S 75
AATCAGAAATTCAAGGACAAGGCCACATTGAATGTAGACAGATCC
S N T A Y M K L S S L T S E D 90
TCCAACACAGCCTACATGAAGCTCAGCAGCCTGACATCTGAGGAC
                                                  CDR3
S A V Y Y C A R T G F D Y W G 105
TCTGCAGTCTATTACTGTGCAAGAACTGGTTTTGACTACTGGGGC
Q G T T L T V S S 114
CAAGGCACCACTCTCACAGTCTCCTCA
```

TABLE 17

| Seq ID | | | |
|---|---|---|---|
| No. | Name | Ab region | Type |
| 1 | 1181 | LCVR | polynucleotide |
| 2 | 1621 | LCVR | polynucleotide |
| 3 | 1181 | LCVR | amino acid |
| 4 | 1621 | LCVR | amino acid |
| 5 | 1181 | LCVR CDR1 | amino acid |
| 6 | 1621 | LCVR CDR1 | amino acid |
| 7 | Generic | LCVR CDR1 | amino acid |
| 8 | 1181, 1621, | LCVR CDR2 | amino acid |
| | 4641 & 1451 | | |
| 9 | 1181 & 1621 | LCVR CDR3 | amino acid |
| 10 | 1181 | HCVR | polynucleotide |
| 11 | 1621 | HCVR | polynucleotide |
| 12 | 1181 | HCVR | amino acid |
| 13 | 1621 | HCVR | amino acid |
| 14 | 1181 | HCVR CDR1 | amino acid |
| 15 | 1621 | HCVR CDR1 | amino acid |
| 16 | Generic | HCVR CDR1 | amino acid |
| 17 | 1181 & 1621 | HCVR CDR2 | amino acid |
| 18 | 1181 & 1621 | HCVR CDR3 | amino acid |
| 19 | human ghrelin | | amino acid |
| 20 | 4641 | LCVR CDR1 | amino acid |
| 21 | 4641 | LCVR CDR3 | amino acid |
| 22 | 1451 | LCVR CDR1 | amino acid |
| 23 | 1451 | LCVR CDR3 | amino acid |
| 24 | 4641 & 1451 | HCVR CDR1 | amino acid |
| 25 | 4641 | HCVR CDR2 | amino acid |
| 26 | 1451 | HCVR CDR2 | amino acid |
| 27 | 4641 & 1451 | HCVR CDR3 | amino acid |
| 28 | Generic | LCVR CDR1 | amino acid |
| 29 | Generic | LCVR CDR3 | amino acid |
| 30 | 4641 | LCVR | amino acid |
| 31 | 1451 | LCVR | amino acid |
| 32 | 4641 | HCVR | amino acid |
| 33 | 1451 | HCVR | amino acid |
| 34 | 1451 | LCVR | polynucleotide |
| 35 | 1451 | HCVR | polynucleotide |
| 36 | 4641 | LCVR | polynucleotide |
| 37 | 4641 | HCVR | polynucleotide |

TABLE 18

Anti-Ghrelin Fab Sequences
(1181, 1621, 4641, 1451)

SEQ ID NO: 1
Polynucleotide sequence encoding 1181 light chain
variable region (CDR sequences in bold face):
5′GATGTTGTGATGACCCAAACTCCACTCTCCCTGCCTGTCAGTCTT
GGAGATCAAGCCTCCATCTCTTGCAGATCTAGTCAGAGCCTTGTA
CACAGTAATGGAAACACCTATTTACATTGGTACCTGCAGAAGCCA
GGCCAGTCTCCAAAGCTCCTGATCTACAAAGTTTCCAACCGATTT
TCTGGGGTCCCAGACAGGTTCAGTGGCAGTGGATCAGGGACAGAT
TTCACACTCAAGATCAGCAGAGTGGAGGCTGAGGATCTGGGAGTT
TATTTCTGCTCTCAAAGTACACATGTTCCGTACACGTTCGGAGGG
GGGACCAAGCTGGAAATAAAACGG3′
SEQ ID NO: 2
Polynucleotide sequence encoding 1621 light chain
variable Region (CDR sequences in bold face):
5′GATGTTGTGCTGACCCAAACTCCACTCTCCCTGCCTGTCAGTCTT
GGAGATCAAGCCTCCATCTCTTGCAGATCTAGTCAGAGCCTTGTA
CACAGTAATGGAAGCACCTATTTACATTGGTACCTGCAGAAGCCA
GGCCAGTCTCCAAAGCTCCTGATCTACAAAGTTTCCAACCGATTT
TCTGGGGTCCCAGACAGGTTCAGTGGCAGTGGATCAGGGACAGAT
TTCACACTCAAGATCAGCAGAGTGGAGGCTGAGGATCTGGGAGTT
TATTTCTGCTCTCAAAGTACACATGTTCCGTACACGTTCGGAGGG
GGGACCAAGCTGGAAATAAGACGG3′
SEQ ID NO: 3
1181 light chain variable region amino acid
sequence (CDR sequences in bold face):
DVVMTQTPLSLPVSLGDQASISCRSSQSLVHSNGNTYLHWYLQKP
GQSPKLLIYKVSNRFSGVPDRFSGSGSGTDFTLKISRVEAEDLGV
YFCSQSTHVPYTFGGGTKLEIKR
SEQ ID NO: 4
1621 light chain variable region amino acid
sequence (CDR sequences in bold face):
DVVLTQTPLSLPVSLGDQASISCRSSQSLVHSNGSTYLHWYLQKP
GQSPKLLIYKVSNRFSGVPDRFSGSGSGTDFTLKISRVEAEDLGV
YFCSQSTHVPYTFGGGTKLEIKR
SEQ ID NO: 5
1181 light chain CDR1 amino acid sequence:
RSSQSLVHSNGNTYLH
SEQ ID NO: 6
1621 light chain CDR1 amino acid sequence:
RSSQSLVHSNGSTYLH
SEQ ID NO: 7
Light chain (generic for 1181 and 1621) CDR1 amino
acid sequence:
RSSQSLVHSNGX₁₂TYLH
wherein X₁₂ is selected from the group
consisting of Gly (G), Ala (A), Ser (S), Thr (T),
Cys (C), Asn (N) and Gln (Q)
SEQ ID NO: 8
1181 and 1621 light chain CDR2 amino acid
sequence:
KVSNRFS
SEQ ID NO: 9
1181 and 1621 light chain CDR3 amino acid
sequence
SQSTHVPYT
SEQ ID NO: 10
Polynucleotide sequence encoding 1181 heavy chain
variable region (CDR sequences in bold face):
5′CAGGTCCAGCTGCAGCAGTCTGGGGCAGAGCTTGTGAGGTCAGGG
GCCTCAGTCAAGTTGTCCTGCACAGCTTCTGGCTTCAACATTAAA
GACTACTTTATGCAGTGGGTGAAGCAGAGGCCTGAACAGGGCCTG
GAGTGGATTGGATGGATTGATCCTGAGAATGGTGAAACTGGATAT
GCCCCGAAGTTCCAGGGCAAGGCCACTATGACTGCAGACACAGCC
TCCAATACAGCCTACCTGCAACTCAGCAGCCTGACATCTGAGGAC
ACTGCCCTGTATTACTGTAATGCACCTTCGGTCGTGGCTAAATAC
TTCGATGTCTGGGGCGCAGGGACCACGGTCACCGTCTCCTCA3′
SEQ ID NO: 11
Polynucleotide sequence encoding 1621 heavy chain
variable region (CDR sequences in bold face):
5′CAGGTCCAGCTGCAGCAGTCTGGGGCAGAGCTTGTGAGGTCAGGG
GCCTCAGTCAAGTTGTCCTGCACAGCTTCTGGCTTCAACATTAAA
GACTACTTTGTGCAGTGGGTGAAGCAGAGGCCTGAACAGGGCCTG
GAGTGGATTGGATGGATTGATCCTGAGAATGGTGAAACTGGATAT
GCCCCGAAGTTCCAGGGCAAGGCCACTATGACTGCAGACACAGCC
TCCAATACAGCCTACCTGCAACTCAGCAGCCTGACATCTGAGGAC
ACTGCCCTGTATTTCTGTAATGCACCTTCGGTAGTGGCTAAATAC
TTCGATGTCTGGGGCGCAGGGACCACGGTCACCGTCTCCTCA3′
SEQ ID NO: 12
1181 heavy chain amino acid sequence (CDR
sequences in bold face):
QVQLQQSGAELVRSGASVKLSCTASGFNIKDYFMQWVKQRPEQGLEWIG
WIDPENGETGYAPKFQGKATMTADTASNTAYLQLSSLTSEDTALYYCNA
PSVVAKYFDVWGAGTTVTVSS
SEQ ID NO: 13
1621 heavy chain amino acid sequence (CDR
sequences in bold face):
QVQLQQSGAELVRSGASVKLSCTASGFNIKDYFVQWVKQRPEQGLEWIG
WIDPENGETGYAPKFQGKATMTADTASNTAYLQLSSLTSEDTALYFCNA
PSVVAKYFDVWGAGTTVTVSS
SEQ ID NO: 14
1181 heavy chain CDR1 amino acid sequence:
GFNIKDYFMQ
SEQ ID NO: 15
1621 heavy chain CDR1 amino acid sequence:
GFNIKDYFVQ
SEQ ID NO: 16
Heavy chain CDR1 amino acid sequence (generic for
1181 and 1621):
GFNIKDYFX₉Q
wherein X₉ is a hydrophobic amino acid
selected from the group consisting of Val (V),
Leu (L), Ile (I), Met (M) and Pro (P)
SEQ ID NO: 17
1181 and 1621 heavy chain CDR2 amino acid
sequence:
WIDPENGETGYAPKFQG
SEQ ID NO: 18
1181 and 1621 heavy chain CDR3 amino acid
sequence:
PSVVAKYFDV
SEQ ID NO: 19
Human Ghrelin amino acid sequence:
GSSFLSPEHQRVQQRKESKKPPAKLQPX₂₈
wherein X₂₈ is Arg (R) or absent
SEQ ID NO: 20
4641 light chain CDR1 amino acid sequence:
RSSQSLVHSDGNTYLD
SEQ ID NO: 21
4641 light chain CDR3 amino acid sequence:
SQSTYVSYT
SEQ ID NO: 22
1451 light chain CDR1 amino acid sequence:
RSSQSLVHSNGNTYLH
SEQ ID NO: 23
1451 light chain CDR3 amino acid sequence:
SQSTLVPYT
SEQ ID NO: 24
4641 and 1451 heavy chain CDR1 amino acid
sequence:
AYTFTTYWMH
SEQ ID NO: 25
4641 heavy chain CDR2 amino acid sequence:
MIDPSNSDTWLNQKFD
SEQ ID NO: 26
1451 heavy chain CDR2 amino acid sequence:
MIDPYNSETWLNQKFKD
SEQ ID NO: 27
4641 and 1451 heavy chain CDR3 amino acid
sequence:
TGFDY
SEQ ID NO: 28
Light chain (generic for 4641, 1451, 1181 and
1621) CDR1 amino acid sequence:
RSSQSLVHSX₁₀GX₁₂TYLX₁₆
wherein X₁₀ is selected from the group consisting
of Asn (N) and Asp (D); 42 is selected from the
group consisting of Gly (G), Ala (A), Ser (S),
Thr (T), Cys (C), Asn (N) and Gln (Q); and X₁₆
is selected from the group consisting of His (H)
and Asp (D).
SEQ ID NO: 29
Light chain (generic for 4641, 1451, 1181 and
1621) CDR3 amino acid sequence:
SQSTX₅VX₇YT
wherein X₅ is selected from the group consisting
of His (H), Tyr (Y), and Leu (L); and X₇ is
selected from the group consisting of Pro (P),
Ser (S) and Thr (T).
SEQ ID NO: 30
4641 light chain variable region amino acid
sequence (CDR sequences in bold face):
DVVMTQTPLSLPVSLGDQASISCRSSQSLVHSDGNTYLHWYLQKPGQSPK
LLIYKVSNRFSGVPDRFSGSGSGTDFTLKISRVEAEDLGVYFCSQSTYVS
YTFGGGTKLEIKR
SEQ ID NO: 31
1451 light chain variable region amino acid
sequence (CDR sequences in bold face):
DVVLTQTPLSLPVSLGDQASISCRSSQSLVHSNGNTYLHWYLQKPGQSPK
LLIYKVSNRFSGVPDRFSGSGSGTDFTLKISRVEAEDLGVYFCSQSTHVP
YTFGGGTKLEIKR
SEQ ID NO: 32
4641 heavy chain variable region amino acid
sequence (CDR sequences in bold face)
QVQLQQSRPELVRPGASVKMSCRASAYTFTTYWMHWVRQRPGQGLEWIGM
IDPSNSDTWLNQKFKDKATLNVDRSSNTAYMQLTSLTSEDSAVYYCARTG
FDYWGQGTTLTVSS
SEQ ID NO: 33
1451 heavy chain variable region amino acid
sequence (CDR sequences in bold face):
QVHVKQSGPELVRPGASVKMSCKASAYTFTTYWMHWVKQRPGQGLEWIGM
IDPYNSETWLNQKFKDKATLNVDRSSNTAYMKLSSLTSEDSAVYYCARTG
FDYWGQGTTLTVSS
SEQ ID NO: 34
1451 LCVR polynucleotide
tctactccagcttgggcagatgttgtgctgacccaaactccactctccct
gcctgtcagtcttggagatcaagcctccatctcttgcagatctagtcaga
gccttgtacacagtaatggaaacacctatttacattggtacctgcagaag
ccaggccagtctccaaagctcctgatctacaaagtttccaaccgattttc
tggggtcccagacaggttcagtggcagtggatcagggacagatttcacac
tcaagatcagcagagtggaggctgaggatctgggagtttatttctgctct
caaagtacacatgttccgtacacgttcggtggaggcaccaagctggaaat
caaacgggctgatgcttcaccaactGTA
SEQ ID NO: 35
1451 HCVR polynucleotide
caggtccacgtgaagcagtctgggcctgaactggtgaggcctggggcttc
agtgaagatgtcctgcaaggcttcagcctataccttcaccacctactgga
tgcactgggtgaaacagaggcctggacaaggccttgagtggattggcatg
attgatccttacaatagtgaaacttggttaaatcagaaattcaaggacaa
ggccacattgaatgtagacagatcctccaacacagcctacatgaagctca
gcagcctgacatctgaggactctgcagtctattactgtgcaagaactggt
tttgactactggggccaaggcaccactctcacagtctcctcagccaaaac
gacaccccca
SEQ ID NO: 36
4641 HCVR polynucleotide
tctactccagcttgggcagatgttgtgatgacccaaactccactctccct
gcctgtcagtcttggagatcaagcctccatctcttgcagatctagtcaga
gccttgtacacagtgatggaaacacctatttacattggtacctgcagaag
ccaggccagtctccaaagctcctgatctacaaagtttccaaccgattttc
tggggtcccagacaggttcagtggcagtggatcagggacagatttcacac
tcaagatcagcagagtggaggctgaggatctgggagtttatttctgctct
caaagtacatatgtttcgtacacgttcggaggggggaccaagctggaaat
aaaacgggctgatgctgcaccaactgta
SEQ ID NO: 37
4641 HCVR polynucleotide
caggtccagctgcagcagtctcggcctgaactggtgaggcctggggcttc
agtgaagatgtcctgcagggcttcagcctataccttcaccacctactgga
tgcactgggtgagacagaggcctggacaaggccttgagtggattggcatg
attgatccttccaatagtgatacttggttaaatcagaagttcaaggacaa
ggccacattgaatgtagacagatcctccaacacagcctacatgcagctca
ccagcctgacatctgaggactctgcagtctattactgtgcaagaactggt
tttgactactggggccaaggcaccactctcacagtctcctcagccaaaac
gacaccccca

1-40. (canceled)

41. A monoclonal antibody or antigen-binding portion thereof, wherein:
a) the light chain variable region comprises a peptide with the sequence shown in SEQ ID NO:3 and the heavy chain variable region comprises a peptide with the sequence shown in SEQ ID NO:12;
b) the light chain variable region comprises a peptide with the sequence shown in SEQ ID NO:4 and the heavy chain variable region comprises a peptide with the sequence shown in SEQ ID NO:13;
c) the light chain variable region comprises a peptide with the sequence shown in SEQ ID NO:30 and the heavy chain variable region comprises a peptide with the sequence shown in SEQ ID NO:32; or
d) the light chain variable region comprises a peptide with the sequence shown in SEQ ID NO:31 and the heavy chain variable region comprises a peptide with the sequence shown in SEQ ID NO:33.

42. The monoclonal antibody or antigen-binding portion thereof of claim 41, which comprises a heavy chain constant region selected from the group consisting of IgG₁, IgG₂, IgG₃, IgG₄, IgA, IgE, IgM, and IgD.

43. The monoclonal antibody or antigen-binding portion thereof of claim 41, which comprises a kappa or lambda light chain constant region.

44. The monoclonal antibody or antigen-binding portion thereof of claim 41, which is a Fab fragment, a F(ab′)₂ fragment, or a single chain Fv fragment.

45. The monoclonal antibody or antigen-binding portion thereof of claim 41, wherein a CDR has 1 or 2 conservative amino acid substitutions or terminal deletions.

46. The monoclonal antibody or antigen-binding portion thereof of claim 41, which is chimeric.

47. A pharmaceutical composition, comprising the chimeric monoclonal antibody or antigen-binding portion thereof of claim 46, and a pharmaceutically acceptable carrier, diluent, or excipient.

48. A method of treating obesity or a related disorder in a mammal, comprising administering to a patient in need thereof an effective amount of a chimeric monoclonal antibody or antigen-binding portion thereof of claim 46.

49. The method of claim 48, wherein said related disorder is selected from the group consisting of NIDDM, Prader-Willi syndrome, an eating disorder, hyperphagia, impaired satiety, anxiety, and a gastric motility disorder.

50. The method of claim 48, wherein said mammal is a human.

51. The method of claim 49, wherein said mammal is a human.
